(19) United States
(12) Patent Application Publication
(10) Pub. No.: US 2023/0288424 A1
(43) Pub. Date: Sep. 14, 2023

(54) BIOMARKERS FOR IDENTIFYING MPMRI VISABLE TUMOURS AND ASSESSING TUMOUR AGGRESSIVENESS OF PROSTATE CANCER

(71) Applicants: University Health Network, Toronto (CA); The Regents of the University of California, Oakland, CA (US)

(72) Inventors: Thomas Kislinger, Toronto (CA); Paul C. Boutros, Toronto (CA); Amanda Khoo, Toronto (CA); Yiyang Liu, Toronto (CA)

(21) Appl. No.: 18/116,963

(22) Filed: Mar. 3, 2023

Related U.S. Application Data
(60) Provisional application No. 63/316,093, filed on Mar. 3, 2022.

(51) Int. Cl. G01N 33/574; C12Q 1/6886

(52) U.S. Cl. CPC G01N 33/57434; C12Q 1/6886; G01N 2800/52; C12Q 2600/158

(57) ABSTRACT

The present disclosure provides methods of prognosis of prostate cancer using mpMRl visibility biomarkers selected from SRD5A2, GNA11, CAPNS1, NCDN, WDR5 and/or LDHB.

### RELATED APPLICATION

This application claims the benefit of U.S. provisional application Ser. No. 63/316,093 filed Mar. 3, 2022, the entire contents of which are hereby incorporated by reference.

### GOVERNMENT FUNDING

This invention was made with government support under Grant Number CA214194 and CA016042, awarded by the National Institutes of Health. The US government has certain rights in the invention.

### FIELD

The present disclosure relates to the use of biomarkers to predict mpMRI visibility of prostate cancer.

### BACKGROUND

Multiparametric magnetic resonance imaging (mpMRI) has improved the management of localized prostate cancer, but 20% of clinically significant tumours are invisible to it 1. mpMRI could be used to reduce unnecessary needle biopsies^{2,3}, but given this false-negative rate there is limited consensus on which men suspected of prostate cancer with negative mpMRI can safely avoid them⁴.

The biological processes that drive mpMRI invisibility are largely unknown, despite clinical differences in their presentation. Understanding the molecular differences in visible and invisible tumours could help to identify invisible tumours that are likely clinically aggressive. mpMRI visibility is associated with nimbosus⁵, a constellation of genomic, transcriptomic and histopathological features that signal aggressive prostate cancers. These include increased genomic instability, presence of intraductal carcinoma and/or cribriform architecture histology (IDC/CA), expression of long non-coding RNA SChLAP1, and hypoxias. Elevated hypoxia in visible tumours suggests that the tumour microenvironment might play a role in tumour visibility on mpMRI⁷, possibly due to differences in stromal organization⁸ that could lead to restricted water diffusion. mpMRl visibility is also associated with a signature of 7 RNAs: SNORA12, SNORA54, SNORD68, SNORD3A, SNORD33, SNORA37 and SCARNA5.⁵

mpMRl is routinely performed on prostate cancer patients whose tumour is categorized as International Society of Urological Pathology (ISUP) Grade Group 2 or above. For prostate cancer patients whose tumour is categorized as ISUP Grade Group 1, the standard of care is active surveillance. For prostate cancer patients whose tumour is categorized as ISUP Grade Group 2, however, there is little consensus on whether mpMRl should be performed because of the 20% false-negative rate and the delays, costs, and inter-observer variability associated with mpMRIs. Accordingly, mechanisms to more accurately and/or easily identify tumours that will be visible to mpMRl are desirable.

### SUMMARY OF THE DISCLOSURE

Multiparametric magnetic resonance imaging (mpMRl) has improved the diagnosis and risk-stratification of localized prostate cancer. About 20% of clinically significant tumours are invisible to mpMRl, defined as a PI-RADSv2 score of one or two. To understand the determinants of mpMRl visibility, the proteomes of twenty mpMRl-visible and twenty mpMRl-invisible ISUP Grade Group 2 tumours, along with histologically normal prostate adjacent to the tumour, were examined. Differences in the proteome of tumours, but not stroma, were associated with mpMRl-visibility. The proteomes of mpMRl-invisible tumours were more similar to that of histologically normal prostate mpMRl. It is demonstrated herein that mpMRl visibility can be predicted by a three-protein biomarker (AUC=0.88, 95% CI=0.77-0.98), and this signature is associated with the poor outcome of biochemical relapse after definitive local therapy.

An aspect of the present disclosure provides a method of identifying a prostate cancer tumour that is likely mpMRl visible in a subject, the method comprising obtaining a sample collected from the subject; measuring a polypeptide level of one or more mpMRl visibility biomarkers selected from SRD5A2, GNA11, CAPNS1, NCDN, WDR5 and/or LDHB, in the sample collected from the subject; wherein the level of the one or more mpMRl visibility biomarkers is indicative of mpMRl visibility of the tumour.

Another aspect of the present disclosure provides a method of selecting a subject with or suspected of having prostate cancer for mpMRl, the method comprising determining a polypeptide level of one or more mpMRl visibility biomarkers and selecting the subject for mpMRl when the level of the one or more mpMRl visibility biomarkers indicates that the tumour is mpMRl visible.

In some embodiments, the method further comprises performing mpMRl.

In some embodiments, the method further comprises repeating the method after an interval.

Another aspect of the present disclosure provides a method for prognosing or monitoring aggressiveness of a prostate cancer tumour in a subject, comprising determining a polypeptide level of one or more mpMRl visibility biomarkers, wherein the level of the one or more mpMRl visibility biomarkers is indicative of the prognosis of the subject.

In an embodiment, the prognosing comprises predicting an increased risk of biochemical relapse, wherein the subject is a subject that has undergone treatment.

In an embodiment, the method further comprises treating a patient determined to be at increased risk of biochemical relapse.

Another aspect of the present disclosure provides a use of the measure polypeptide level of the one or more mpMRl visibility biomarkers in a prostate sample collected from a subject with or suspected of having a prostate cancer tumour for selecting a suitable treatment plan and risk stratification.

In an embodiment, the prostate sample is a treatment-naïve tumour sample.

In an embodiment, the prostate sample is a tumour sample taken from a treatment-naïve subject.

Yet another aspect of the present disclosure provides a kit comprising at least two binding agents, each specific for a polypeptide selected from the mpMRl visibility biomarkers disclosed herein.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIGS. 1A-1I are results of proteomic analysis of mpMRl visibility. A) Outline of the samples used for proteomics, RNA-sequencing and copy number analysis. B) Summary of the 4,772 protein groups detected and the distribution of median protein abundance versus the number of samples each protein was detected in, and colored by protein abundance deciles. Top barplot shows the total number of proteins quantified in various number of samples. C) Differentially abundant proteins in mpMRl-visible and invisible normal tissue adjacent to the tumour (NAT) were determined using Mann-Whitney U-test. There were no statistically significant proteins. D) Differentially abundant proteins in tumour and NAT regions were determined using a paired Mann-Whitney U-test. Proteins with FDR<0.05 are colored in black. Protein counts in the bottom corners denote the number of significant proteins (FDR<0.05) in each group. Proteins of interest are marked by a cross. E) Correlation between protein and RNA when comparing tumour-NAT differences. Genes that were significantly associated with tumours or NATs at both RNA and protein levels (FDR<0.05) and had the same directionality are colored in black. p: Spearman's rho; P: Spearman's correlation p value. F) Differentially abundant proteins in mpMRl-visible and invisible tumours were determined using Mann-Whitney U-test. Statistically significant proteins (FDR<0.2) are colored in black. G) Associations of protein abundance changes between tumour versus NAT (n=80), and mpMRl-visible tumour versus mpMRI-invisible tumour (n=40). Proteins that were significant in the tumour-NAT comparison are marked in black. p: Spearman's rho; Pp: Spearman's correlation p value; nᵤₚ: Proteins significantly enriched in tumour (log 2 fold change >0 and p<0.05) and enriched in visible tumour (log 2 fold change >0); n_{down}: Proteins significantly enriched in NAT (log 2 fold change <0 and p<0.05) and enriched in visible tumour (log 2 fold change <0). H) Distribution of Euclidean distance between each group and median protein abundance in NAT (n=40). Top panel: Distance from NAT to NAT centroid (n=40), from invisible tumour (n=20) or from visible tumour (n=20). Difference in the median distance of invisible and visible tumour groups was determined using a Mann-Whitney U-test, with unadjusted p-values. Middle panel: Distance between NAT (n=40), IDC/CA-tumour (n=29), and IDC/CA+ tumour (n=11) to the NAT centroid. Bottom panel: Distance between NAT centroid and NATs (n=40), non-hypoxic (n=20) and hypoxic tumours (n=20). I) Pre-ranked gene set enrichment analysis was performed using hallmark gene sets for the 3 sets of comparisons (Tumour/NAT, visible/invisible tumour, and visible/invisible NAT). Genes were ranked by protein effect size. The dot size denotes the normalized enrichment score (NES) while the dot color denotes if the NES was positive (light grey) or negative (dark grey). The background shading denotes significance. cISUP: clinical International Society of Urological Pathology grade group; mpMRl: multiparametric magnetic resonance imaging; PI-RADS: Prostate Imaging-Reporting and Data System; CNA: copy number analysis; P: nominal p-value; FC: fold change; NAT: Normal adjacent tumour; IDC/CA: intraductal carcinoma or cribriform architecture; FDR: false discovery rate; NES: normalized enrichment score.

FIGS. 2A-2E show protein associations with genomic, transcriptomic and pathological hallmarks of mpMRl visibility. A) Heatmaps summarizing the 14,044 protein-coding RNAs (left) and 1,622 proteins (right) associated with hallmarks of mpMRl-visibility. Associations between the expression of protein-coding RNAs and each hallmark in the discovery cohort^{9,17} (n=144) were determined using Spearman's correlation or log 2 fold change for continuous (snoRNAs, Hypoxia, SChLAP1, and PGA) and binary (IDC/CA) variables, respectively. Statistically significant RNAs (FDR<0.2) from the discovery cohort were validated in matched transcriptomic profiling from our cohort (n=40)⁵. The heatmap shows the effect size of the 14,044 validated RNAs that were associated with at least one hallmark in the validation cohort, colored by positive (light grey) or negative (dark grey) associations. RNAs that were associated with at least one hallmark were used to identify proteins that were associated with each hallmark. Proteins that validated (FDR<0.2 and had the same directionality in the RNA data) are shown in the right heatmap. Top barplot shows the number of hallmarks each RNA or protein was associated with. Side barplot shows the number of RNAs or proteins associated with each hallmark. Bottom covariate bar indicates significant RNAs or proteins (FDR<0.2) associated with visible (light grey) or invisible (dark grey) tumours. Cutout of the right covariate bar at the bottom highlights 3 proteins that were associated with at least one hallmark and mpMRl-visibility. PGA: percent genome altered; IDC/CA: intraductal carcinoma or cribriform architecture. B) Genes that were associated with three or more hallmarks or mpMRl-visibility at the protein level. Left barplot shows the number of hallmarks each gene is associated with at the RNA (light grey) or protein (dark grey) level. Dotmaps show the effect size of the correlation between gene expression and each hallmark. The size of the dot represents the magnitude of the effect, the color denotes the direction, and background shading the FDR. Right barplot shows the log 2 fold change between visible and invisible tumour for RNA (light grey) and protein (dark grey), with significant differences marked by asterisks (Mann-Whitney U-test). C) Spearman's rank correlation between protein-PGA associations and protein-MRI-visibility associations (n=4,772 proteins). Validated proteins whose protein abundance was significantly correlated with PGA (FDR<0.2) are colored in black. D) Summary of the correlation between associations with each hallmark and mpMRl-visibility in protein-coding RNAs and proteins. E) A three-protein signature accurately predicted mpMRl-visible tumours with AUC 88%. Confidence intervals shaded in light grey. Inset: The protein signature was associated with worse biochemical recurrence (BCR)-free survival in an independent cohort⁹ (n=76 patients). Low: n=49, 20 events; High: n=26, 15 events. FDR: false discovery rate; p: Spearman's rho; FC: fold change; HR: hazard ratio.

FIGS. 3A-3G are plots and a heatmap showing the numbers of proteins quantified. Boxplot of the number of proteins quantified in NAT and Tumour in A) all patients (n=40). B) patients with invisible tumours (n=20), or C) patients with visible tumours (n=20). P-value from paired Mann-Whitney U-test is shown. D) Consensus clustering of samples (n=81, K=4) using the top 25% most variable proteins (n=1,193, K=4). E) Differentially abundant protein-coding RNAs in tumours and NATs from TCGA were determined using Mann-Whitney U-test. Statistically significant proteins (FDR<0.2) are colored in black. F) Associations of protein abundance changes between tumor versus NAT, and mpMRI-visible NAT versus mpMRI-invisible NAT G) Associations of protein-coding RNA abundance changes between tumour versus NAT (nₜᵤₘₒᵤᵣ=499, n_{NAT}=53), and mpMRI-visible tumour versus mpMRI-invisible tumour (n=40). Proteins that were significant in the tumour-NAT comparison are marked in black. p: Spearman's rho; Pp: Spearman's correlation p value; nᵤₚ: Proteins significantly enriched in tumour (log 2 fold change >0 and p<0.05) and enriched in visible tumour (log 2 fold change >0); n_{down}: Proteins significantly enriched in NAT (log 2 fold change <0 and p<0.05) and enriched in visible tumour (log 2 fold change <0).

FIGS. 4A-4B are plots showing the predictive value of combined signatures. A) Model combining our 3-protein signature and nimbosus hallmarks (PGA, Schlap1, hypoxia, and IDC/CA) predicts mpMRI-visible tumours with AUC 82%. B) Model combining our 3-protein signature and a snoRNA signatures predicts mpMRI-visible tumours with AUC 83%. Confidence intervals shaded in light grey.

### DETAILED DESCRIPTION OF THE DISCLOSURE

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. Definitions

As used herein, the term “mpMRI visibility” refers to a characterization of localized prostate cancer based on mpMRI evaluation of the tumour. Prostate tumours that are identified as invisible, are typically less aggressive and/or clinically significant and tumours identified as visible are typically more aggressive and/or clinically significant. For example, the tumour may be evaluated following the categories defined by the Prostate Imaging—Reporting and Data System (PI-RADS) version 2. PI-RAD v2 uses a 5-point scale in assessing the presence of a clinically significant tumour in the prostate gland based on mpMRl findings. The lower the category, the lower the likelihood that clinically significant cancer is present. The terms “mpMRl-invisible”, “invisible to mpMRl” and the like refer to a tumour that can be categorized as or that corresponds to PI-RADS v2 category 1 or category 2 using the PI-RADS scale. The terms “mpMRl-visible”, “visible to mpMRl” and the like refer to a tumour that is categorized as or corresponds to PI-RADS v2 category 3, 4 or 5.

“Clinically significant tumour” as used herein refers to a tumor that can surgically defined as Gleason score 7 or greater, tumor volume of 0.5 cm³ or greater, or tumour category T3 or greater (Seo J W et al. AJR Am J Roentgenol. 2017;209:W1-W9).

As used herein, the term “grade” refers to categorization of a tumour based on the aggressiveness of the tumour or the likelihood that the tumour will grow and spread. For example, the classical grading system for prostate cancer is the Gleason score, where a Gleason score of 6 or less indicates a low likelihood of the tumour metastasizing. Another commonly used grading system is the ISUP (International Society of Urological Pathology) system, where Grade Group 1 is the least aggressive and Grade Group 5 is the most aggressive. Patients with Grade Group 1 tumours are typically not treated with surgery, radiation or hormone therapy, and are monitored by active surveillance. Patients with Grade Group 2 are treated with surgery or radiation, although there is an increasing body of evidence suggesting that some patients with Grade Group 2 tumors may not need surgical or radiological intervention and would benefit from active surveillance as their tumors are less aggressive (Carlsson S et al. J Urol. 2020; 203:1117-1121.).A subset of these patients with Grade Group 2 tumors however have tumours that will metastasize and therefore would have benefited from treatment.

As used herein, the term “prognosing” refers to predicting the development of a disease, outcome of a treatment and/or a procedure in relation to the disease. Prognosis may be assessed on a variety of bases, including but not limited to biomarkers, clinical data, genetics etc.

As used herein, the term “biomarker” refers to any molecules, including but not limited to proteins, polypeptides, nucleic acids such as mRNA, lipids, metabolites, modifications thereof, that can be used as an indicator of a biological state, in the diagnosis/prognosis of a disease or disorder, and/or in the prediction of the outcome of a treatment or procedure. A biomarker may be used on its own, or in combination with other biomarkers and/or methods.

As used herein, the term “mpMRl visibility biomarker” refers to any biomarker described herein that can be used to predict whether a prostate cancer is mpMRl visible. As used herein, the term refers to any of the following: SRD5A2, GNA11, CAPNS1, NCDN, WDR5 and/or LDHB. A mpMRl visibility biomarker may be associated with mpMRl-visible tumour or with mpMRl-invisible tumour. For example, a mpMRl visibility biomarker can be detected in both mpMRl-visible tumour and mpMRl-invisible tumour but differentially expressed between them. As another example, a mpMRl visibility biomarker can only be detected in mpMRl-visible tumour. As another example, a mpMRl visibility biomarker can only be detected in mpMRl-invisible tumour.

The term “SRD5A2” refers to the protein 3-oxo-5-alpha-steroid 4-dehydrogenase 2 and encompasses variants, isoforms, mutant forms etc. The term also encompasses homologues in different species.

The term “GNA11” refers to the protein guanine nucleotide-binding protein subunit alpha-11 and encompasses variants, isoforms, mutant forms etc. The term also encompasses homologues in different species.

The term “CAPNS1” refers to the protein calpain small subunit 1 and encompasses variants, isoforms, mutant forms etc. The term also encompasses homologues in different species.

The term “NCDN” refers to the protein Neurochondrin and encompasses variants, isoforms, mutant forms etc. The term also encompasses homologues in different species.

The term “WDR5” refers to the protein WD repeat-containing protein 5 and encompasses variants, isoforms, mutant forms etc. The term also encompasses homologues in different species.

The term “LDHB” refers to the protein lactate dehydrogenase B and encompasses variants, isoforms, mutant forms etc. The term also encompasses homologues in different species.

As used herein, the term “PGA” refers to the proportion of genome altered by copy number aberrations (changes in copy number in reference to the diploid human genome) and is used as a measure of genomic instability. Proportion of genome altered is calculated by summing the total number of bases covered by the copy number aberrations divided by the total size of the whole genome. It is one of the hallmarks of mpMRl-visible tumours.

As used herein, the term “IDC/CA” means intraductal carcinoma or cribriform architecture, which represent unfavorable sub-histopathologies in localized prostate cancer as described in Chua et al, Eur Urol. 2017; 72:665-674.

As used herein, the terms “biochemical relapse”, “biochemical failure” and “biochemical recurrence” refer to a rise in PSA level in a prostate cancer subject after treatment and may be defined as a PSA level >0.2 ng/ml following radical prostatectomy and >2 ng/ml above the nadir after radiation therapy (Cornford et al. European Urology. 2017; 71:630-42).

As used herein, the terms “level”, “expression level” and the like when used in the context of a biomarker refers to the amount of the biomarker measured/detected in a sample. For example, the level of a protein biomarker refers to the measured/detected amount of the protein.

As used herein, the term “expression data” refers to data comprising information for determining the level of the one or more mpMRl visibility biomarkers. Expression data may be in any form. For example, it may be raw data or processed data; it may comprise different formats such as image files, spreadsheets etc.

As used herein, the terms “polypeptide” and “protein” refer to any chain of two or more natural or unnatural amino acid residues, regardless of post-translational modifications (e.g., glycosylation or phosphorylation).

The term “subject” also interchangeably referred to as patient, as used herein includes all members of the animal kingdom including mammals, and suitably refers to a human.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 20%, 5-20%, or 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be under-stood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Although any methods and materials similar or equivalent to those described herein can also be used in the practice or testing of the present disclosure, examples of methods and materials are now described.

### II. Methods

Multiparametric magnetic resonance imaging (mpMRI) is a valuable tool for the assessment of prostate cancer. Clinical applications of mpMRI include tumour detection, characterization, risk stratification, surveillance, and others. However, about 20% of clinically significant tumours are invisible to mpMRI.

Using a proteomics approach, it is demonstrated herein that unexpectedly, there is no difference between the histologically normal tissue adjacent to the tumour (NAT) of mpMRI-visible and mpMRI-invisible tumours. However, five proteins are differentially abundant between mpMRI-invisible and mpMRI-invisible tumours: SRD5A2, GNA11, CAPNS1, NCDN, WDR5 and/or LDHB. Three of the proteins, SRD5A2, GNA11 and WDR5 are associated with mpMRI visibility, PGA and IDC/CA. It is further demonstrated herein by machine-learning that a panel of three proteins, LDHB, GNA11 and SRD5A2, can predict mpMRI visibility.

Performing mpMRI on a subject whose tumour is mpMRI-invisible is undesirable because resources are wasted. Accordingly, provided herein are methods to predict mpMRI visibility of a tumour, comprising measuring a level of one or more mpMRI visibility biomarkers in sample obtained from the subject, wherein the one or more mpMRI visibility biomarkers is indicative of mpMRI visibility.

Prostate cancer is stratified into different grades that indicate the likelihood that the tumour will grow and/or spread. Where the likelihood is low, standard of care generally involves surveillance. The method disclosed herein can be used to aid in the determination of whether a subject diagnosed with low grade prostate cancer should be assessed by mpMRI. In one embodiment, the subject is a prostate cancer subject whose tumour is categorized as ISUP Grade 1. In another embodiment, the subject is a prostate cancer subject whose tumour is categorized as ISUP Grade 2. Other grading systems and categorization of prostate cancer may be used. For example, the T category of the tumour, serum PSA levels, Gleason score, and the combination thereof. Where a prostate cancer subject whose tumour is categorized as low grade (for example, ISUP Grade 1 or Grade 2) but predicted to be mpMRl-visible, assessment by mpMRl can be beneficial.

mpMRl-visible tumours are associated with genome instability (as indicated by PGA) and unfavourable histology (IDC/CA), and an increased risk of biochemical relapse and metastasis (Houlahan et al, Eur Urol. 2019; 76:18-23).

The methods disclosed herein can have a variety of uses in prostate cancer management including but not limited to selecting a suitable treatment plan and risk stratification.

In one aspect, the present disclosure provides methods for identifying tumour that is likely mpMRl visible in a subject. In some embodiments, the methods may comprise measuring a polypeptide level of one or more mpMRl visibility biomarkers in sample obtained from the subject, wherein the one or more mpMRl visibility biomarkers is indicative of mpMRl visibility. In some embodiments, the methods may comprise obtaining expression data for determining a polypeptide level of one or more mpMRl visibility biomarkers in the sample collected from the subject.

In some embodiments, the methods further comprise selecting the subject for mpMRl if the polypeptide level of the one or more mpMRl visibility biomarkers is indicative that the tumour is visible.

In some embodiments, the methods further comprise performing mpMRl.

mpMRl may be performed, for example, in a 3-T scanner with anti-peristaltic agent, with or without endorectal coil. Parameters can include for example T2 weighted image, dynamic contrast-enhanced image, and diffusion-weighted image. Other parameters can also be used.

In performing mpMRl, clinical guidelines for multi-parametric MRI of the prostate, such as ESUR prostate MR guidelines 2012 (Barentsz, J. O. et al. (2012). ESUR prostate MR guidelines 2012. European radiology, 22(4), 746-757, the content of which is incorporated herein in its entirety) may be followed.

In one aspect, the present disclosure provides methods for selecting a subject with or suspected of having prostate cancer for mpMRl. In some embodiments, the methods may comprise obtaining expression data for determining a polypeptide level of one or more mpMRl visibility biomarkers in the sample collected from the subject. In some embodiments, the methods further comprise performing mpMRl.

In another aspect, the present disclosure provides methods for prognosing or monitoring aggressiveness of a prostate cancer tumour in a subject. In some embodiments, the methods may comprise measuring a polypeptide level of one or more mpMRl visibility biomarkers in sample obtained from the subject, wherein the one or more mpMRl visibility biomarkers is indicative of mpMRl visibility. In some embodiments, the methods may comprise obtaining expression data for determining a polypeptide level of one or more mpMRl visibility biomarkers in the sample collected from the subject.

Prognosing is used as a broad sense and encompasses predicting disease progression such as risk of metastasis, risk of relapse, among others. In an embodiment, prognosing comprises predicting an increased risk of biochemical relapse in a subject that has undergone treatment.

In some embodiments, the methods further comprise treating a subject determined to be at an increased risk of biochemical relapse.

Using the methods disclosed herein, it may be determined that a subject is not to be selected for mpMRl. The subject may then be put under surveillance. It can be appreciated that it is possible the tumour of the subject may become mpMRl visible and/or aggressive at a later time. Accordingly, in some embodiments, the methods may be repeated for the subject after an interval.

It can be appreciated that the determining the level of the one or more mpMRl visibility biomarkers, the identifying tumours that are likely mpMRl visible, the selecting subjects for mpMRl, the prognosing and monitoring aggressiveness of the tumour may be performed by the same party or by different parties. For example, a physician at a hospital may send patient samples to a third party laboratory, who then generates expression data of the one or more mpMRl visibility biomarkers and provides the expression data to the physician.

Therefore, in some embodiments, the methods comprise obtaining a sample collected from the subject with prostate cancer or suspected of having prostate cancer. In some embodiments, the methods comprise obtaining expression data generated from a sample collected from the subject.

Predicting mpMRl visibility of a tumour from a polypeptide level of the one or more mpMRl visibility biomarkers may be done by any suitable method. In some embodiments, a statistical model is used. Suitable statistical models include but are not limited to logistic regression, linear discriminant analysis, multivariate adaptive regression splines, naïve Bayes, neural network, support vector machine, functional tree, LAD tree, Bayesian network, elastic net regression, and random forest. In some embodiments, the statistical model comprises a logistic regression model. In some embodiments, the statistical model is trained on the polypeptide level of the one or more mpMRl visibility biomarkers in a plurality of tumour samples with known mpMRl visibility. In an embodiment, the polypeptide level is log 2-transformed. In an embodiment, a cutoff value of 0.5 is used.

In some embodiments, predicting mpMRl visibility of a tumour comprises comparing a polypeptide level of the one or more mpMRl visibility biomarkers to a threshold level. The threshold level may be in any form, for example, a cutoff value or a range of values. In some embodiments, a polypeptide level above the threshold is indicative of the tumour visible to mpMRl. In some embodiments, a polypeptide level below the threshold is indicative of the tumour visible to mpMRl.

A threshold level can be determined from a plurality of control samples. In some embodiments, control samples are samples from healthy subjects not diagnosed with prostate cancer. In some embodiments, control samples are NATs. In some embodiments, the threshold level is pre-determined. In some embodiments, control samples and test tumour samples are analyzed concurrently.

In some embodiments, the methods comprise determining the difference in the expression level of the one or more mpMRl visibility biomarkers. In some embodiments, determining the difference in expression level comprises comparing the expression level or transformed expression level of two samples. In an embodiment, the transformed expression level can be a log 2 transformed expression level. In some embodiments, the difference is between a tumour sample and a NAT sample. In some embodiments, the difference is between a tumour sample and a sample from a healthy subject not diagnosed with prostate cancer. In some embodiments, the difference is between a mpMRl visible tumour sample and a mpMRl invisible tumour sample. In some embodiments, the difference is between a NAT sample of a mpMRl visible tumor and a NAT sample of a mpMRl invisible tumour. In one embodiment, the one or more mpMRl visibility biomarkers are selected from SRD5A2, GNA11, CAPNS1, NCDN, WDR5 and/or LDHB.

In one embodiment, the one or more mpMRl visibility biomarkers are at least 2, at least 3, at least 4, or at least 5 mpMRl visibility biomarkers. In one embodiment, the one or more mpMRl visibility biomarkers are the mpMRl visibility biomarkers.

For example, the one of more biomarkers can be 1, 2, 3, 4, 5 or 6 of the mpMRl visibility biomarkers.

In one embodiment, the mpMRl visibility biomarker is or comprises SRD5A2. In another embodiment, the mpMRl visibility biomarker is or comprises GNA11. In another embodiment, the mpMRl visibility biomarker is or comprises CAPNS1. In another embodiment, the mpMRl visibility biomarker is or comprises NCDN. In another embodiment, the mpMRl visibility biomarker is or comprises WDR5. In another embodiment, the mpMRl visibility biomarker is or comprises LDHB. In another embodiment, any one of the mpMRl visibility biomarkers can be combined with any one or more of the other mpMRl visibility biomarker.

The ability of individual mpMRl visibility biomarkers to predict mpMRl visibility is for example shown in Table 3.

In one embodiment, the one or more mpMRl visibility biomarkers comprise LDHB, SRD5A2 and GNA11. In one embodiment, the one or more mpMRl biomarkers comprise LDHB and SRD5A2. In one embodiment, the one or more mpMRl visibility biomarkers comprise SRD5A2 and GNA11. In one embodiment, the one or more mpMRl visibility biomarkers comprise LDHB and GNA11.

In one embodiment, the one or more mpMRl visibility biomarkers comprise CAPNS1 and GNA11. In one embodiment, the one or more mpMRl visibility biomarkers comprise CAPNS1 and SRD5A2. In one embodiment, the one or more mpMRl visibility biomarkers comprise CAPNS1 and LDHB, In one embodiment, the one or more mpMRl visibility biomarkers comprise CAPNS1 and WDR5. In one embodiment, the one or more mpMRl visibility biomarkers comprise CAPNS1 and NCDN. In one embodiment, the one or more mpMRl visibility biomarkers comprise GNA11 and WDR5. In one embodiment, the one or more mpMRl visibility biomarkers comprise GNA11 and NCDN. In one embodiment, the one or more mpMRl visibility biomarkers comprise SRD5A2 and WDR5. In one embodiment, the one or more mpMRl visibility biomarkers comprise SRD5A2 and NCDN. In one embodiment, the one or more mpMRl visibility biomarkers comprise LDNB and WDR5. In one embodiment, the one or more mpMRl visibility biomarkers comprise LDNB and NCDN, In one embodiment, the one or more mpMRl visibility biomarkers comprise WDR5 and NCDN.

The ability for pairs of mpMRl visibility biomarkers to predict mpMRl visibility is for example shown in Table 4.

In some embodiments, the sample is a prostate sample. In an embodiment, the prostate sample is a treatment-naïve tumour sample. In an embodiment, the prostate sample is taken from a treatment-naïve subject with prostate cancer.

In some embodiments, the sample is a prostate cancer biopsy. In an embodiment, the biopsy is a transrectal biopsy. In another embodiment, the biopsy is a transperineal biopsy. In some embodiments, the sample is a tumour tissue core sample.

In some embodiments, the sample is cryopulverized. In some embodiments, the sample is processed for protein extraction. In some embodiments, the proteins are digested. In some embodiments, digested proteins are analyzed by mass spectrometry.

In some embodiments, the level of the one or more biomarkers is measured by measuring protein levels. Any suitable methods for protein level measurement known in the art can be used, including but not limited to affinity-based assays, spectroscopy methods, and blotting methods. Affinity-based assays typically comprise the use of one or more binding agents that bind specifically the protein of interest. A variety of binding agents can be used, including but not limited to antibodies and fragments thereof, ligands, receptors, aptamers, oligonucleotides, and molecularly imprinted polymers. The binding agent may bind the full-length protein or a fragment thereof, an isoform, a pro-protein, a post-translationally modified protein etc.

In general, detecting or measuring the level of a biomarker through an affinity-based method comprises contacting a sample with one or more binding agents that specifically bind the biomarker. Each of the binding agents may comprise a different detectable label to allow detection of different mpMRl visibility biomarkers. Detectable labels and moieties include but not limited to florescent dyes such as FITC, Cy3, Cy5, radioisotopes such as iodine-125, enzymes such as horseradish peroxidase, alkaline phosphatase, β-galactosidase, acetylcholinesterase, and catalase, nanoparticles such as gold nanoparticles. Another example of a detectable label that can be used with a binding agent is an aptamer, as used for example in the SOMAscan proteomics technology. The SOMAscan proteomics technology has been used to bind and quantify various protein targets including for example LDHB 24.

In some embodiments, the binding agent can be directly conjugated to the detectable label or moiety. In other embodiments, the binding agent is not labelled and the biomarker-binding agent complex is detected with a secondary reagent, which is conjugated to a detectable label or moiety. The secondary reagent may bind the mpMRl visibility biomarker or the binding agent. Any suitable methods known in the art for conjugating binding agents to labels and moieties can be used.

The use of antibodies and fragments thereof in assays to measure protein levels is well known in the art. Such assays include, but are not limited to, immunochromatographic assay, enzyme-linked immunosorbent assay (ELISA), immunohistochemistry (IHC), western blotting, radioimmunoassays (RIA), fluorescent immunoassays, the practices of which are well known in the art (see, e.g., Ausubel, Frederick M. Current Protocols in Molecular Biology. New York: John Wiley & Sons, 1994, the content of which is incorporated by reference in its entirety). Another example of an antibody-based assay to measure proteins is the Proximity Extension Assay (PEA).

A person skilled in the art would know that each type of assay can come in different formats and any suitable format can be used with the methods disclosed herein. For example, suitable ELISA formats include but not limited to sandwich ELISA.

Examples of commercially available binding agents that can be used to specifically recognize the mpMRl visibility biomarkers disclosed herein include but are not limited to:

| | Examples of commercially available |
|---|---|
| Biomarker | binding agents |
| SRD5A2 | Polyclonal, MilliporeSigma/Merck KGaA Cat no. |
| | SAB2500988 |
| GNA11 | Polyclonal, Invitrogen Cat no PA5-102653 |
| CAPNS1 | Clone 3C4, MilliporeSigma/Merck KGaA Cat no. |
| | WH0000826M1 |
| NCDN | Polyclonal, MilliporeSigma/Merck KGaA Cat no. |
| | SAB1400440 |
| WDR5 | Clone 2C2, MilliporeSigma/Merck KGaA Cat no, |
| | WH0011091M1 |
| LDHB | Clone 2H6, MilliporeSigma/Merck KGaA Cat no, |
| | WH0003945M1 |

The present disclosure also provides kits comprising at least two binding agents, each specific for a polypeptide selected from SRD5A2, GNA11, CAPNS1, NCDN, WDR5 and/or LDHB.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure.

### EXAMPLES

### Example 1

### Methods

### Patient Cohort and Tumour Sectioning

Patient selection, mpMRI imaging acquisition and interpretation, tissue collection, and sample processing has been previously described⁵. In brief, patients with localized prostate cancer and solitary Gleason score 3+4 lesions >1.5 cm on final surgical pathology and with PI-RADSv2 1-2 (MRI-invisible) or PI-RADSv2 5 (MRI-visible) lesions were selected for molecular profiling by copy number profiling⁵, RNA-seq⁵, and proteomics. Patients underwent mpMRI in a 3-T scanner with anti-peristaltic agent, with or without endorectal coil. mpMRI images were reported by 1 of 3 experienced uroradiologists with 10-18 years of prostate mpMRI experience and a retrospective blinded review of the mpMRI invisible tumours were performed by a single uroradiologist. Tumour and normal tissue adjacent to the tumour (NAT) regions were annotated by a genitourinary pathologist. The relevant areas of tissue were macro-dissected from adjacent 10 μm sections for proteomics analysis.

### Tissue Preparation for Shotgun Proteomics

Each scraped FFPE tissue region was placed in a 1.5 mL conical tube and deparaffinized with xylene as follows. 500 μL of buffer was added to each tube, then samples were vortexed at high speed for 30s, incubated for 5 minutes on an end-over-end nutator at room temperature, centrifuged at 18,000 rcf for 3 min, and the supernatant was discarded. The deparaffinization step was repeated twice. Tissues were then rehydrated with a graded ethanol series (95% ethanol, 90% ethanol, 75% ethanol, 50% ethanol, 25% ethanol, and water). Water was evaporated from each tube using a SpeedVac vacuum concentrator (Thermo) until 100 uL of water remained in each tube. 10 uL of 1M Tris pH 8.0 buffer was added to each sample to a final concentration of 100 mM Tris-HCl, pH 8. Samples were heated at 95° C. for 1 hour to reverse formalin-induced crosslinking, then sonicated on a probe-less ultrasonic sonicator for ten 10 second cycles at 10 Watts per tube (Hielscher VialTweeter).

### Protein Digestion

100 μL 2,2,2-Trifluoroethanol was added to each tube to a final concentration of 50%. 2 pmol of SUC2 protein (Yeast invertase) was added as a digestion control. Disulphide bonds were reduced with 5 mM dithiothreitol, followed by 1 hour incubation at 60° C. Free sulfhydryl groups were alkylated by incubating samples in 25 mM iodoacetamide in the dark for 30 min at room temperature. Samples were diluted 1:5 with 100 mM ammonium bicarbonate with 2 mM CaCl2) (pH 8). Proteins were digested with 2 μg of trypsin/Lys-C enzyme mix (Promega) overnight at 37° C., then an additional 1 μg of trypsin/Lys-C enzyme mix was added in the morning and digestion continued at 37° C. for 1 hour. 1% FA was used to bring the sample pH<2. Peptides were desalted by C18-based solid phase extraction, then lyophilized in a SpeedVac vacuum concentrator. Peptides were solubilized in mass spectrometry-grade water with 0.1% formic acid. Peptide concentration was quantified using a NanoDrop Lite (at 280 nm).

### Shotgun Proteomics

2 μg of peptides was used for LC-MS/MS analysis. Synthetic iRT peptides (Biognosis) were spiked into each sample at a 1:10 ratio prior to data acquisition. LC-MS/MS data was acquired using an Easy nLC 1000 (Thermo) nano-flow liquid chromatography system with a 50 cm EasySpray ES803 column (Thermo) coupled to a Q Exactive HF (Thermo) tandem mass spectrometer. Peptides were separated by reverse phase chromatography using a 4-hour nonlinear chromatographic gradient of 4-48% buffer B (0.1% FA in ACN) at a flow rate of 250 nl/min. Column temperature was kept at 45° C. Mass spectrometry data was acquired in data dependent mode with a top 15 method. MS¹ data was acquired at a resolution of 120,000, AGC target of 1e6, and maximum injection time (maxIT) of 30 ms, while MS² data was acquired at a resolution of 30,000, AGC target of 1e5, and maxIT of 110 ms. Data was searched in MaxQuant (version 1.6.1.0) using a merged UniProt protein sequence database containing human protein sequences from Uniprot (complete human proteome; 2015-01-27, number of sequences 42,842), yeast invertase (Suc2) protein sequences from Uniprot, and iRT synthetic peptide sequences (Biognosis). Searches were performed with a maximum of two missed cleavages, and carbamidomethylation of cysteine as a fixed modification. Variable modifications were set as oxidation at methionine and methylation at lysine. The false discovery rate for the target-decoy search was set to 1% for protein, and peptide levels. Intensity-based absolute quantification (iBAQ), label-free quantitation (LFQ), and match between runs (matching and alignment time windows set as 0.7 and 20 min respectively) were enabled. The proteinGroups.txt file was used for subsequent analysis. Protein groups will be referred to as proteins in the text. Reverse hits were removed, and proteins identified with two or more peptides were carried forward. LFQ intensities were used for protein quantitation. For proteins with missing LFQ values, median-adjusted iBAQ values were used as replacement¹⁸.

### Consensus Clustering of Proteomic Data

Consensus clustering (maxK=6; reps=50; pltem=0.8; pFeature=1; distance=Pearson; innerLinkage=average; finalLinkage=average; ConsensusClusterPlus v1.52.0) was performed using divisive hierarchical clustering on the 25% most variable proteins in the cohort to cluster samples and proteins. Missing values were imputed with random values drawn from a normal distribution of protein abundances (width=0.2; down-shift=1.8)¹⁹. Adjusted Rand Index (ARI) (CrossClustering v.4.0.3) was calculated between sample subtypes generated from consensus clustering and two other subtypes: Tumour and NAT, or samples from patients with mpMRl-visible versus mpMRl-invisible tumours.

Differences in Proteins Detected

Mann-Whitney U-test for all comparisons—Tumour vs NAT using all samples, visible tumours only, or invisible tumours only. A linear mixed model with random effects: Imer(count ˜tumour*visible+(1 (subject)); REML=FALSE; ImerTest v.3.1-3) was used to test for independence between tumour vs. NAT (tumour) and invisible vs. visible (visibility) groups (estimateₜᵤₘₒᵤᵣ=−113.26, Pₜᵤₘₒᵤᵣ=1.33×10⁻⁹; estimate_{visibility}=−17.91, P_{visibility}=0.268; estimate_{tumourvisibility}=−8.16, P_{tumourvisibility}=0.570).

Differential Abundance Analysis

For each comparison, proteins present in >50% of the samples were kept for further analysis—visible versus invisible NAT (nₛₐₘₚₗₑₛ=40, nₚᵣₒₜₑᵢₙₛ=4,165), tumour versus NAT (nₛₐₘₚₗₑₛ=80, nₚᵣₒₜₑᵢₙₛ=4,314), visible versus invisible tumour (nₛₐₘₚₗₑₛ=40, nₚᵣₒₜₑᵢₙₛ=4,426). Proteins in the intersection of these 3 sets (nₚᵣₒₜₑᵢₙₛ=4,067) were used for differential expression analysis using the Mann-Whitney U-test, with multiple testing correction using the Benjamini-Hochberg method. Missing values were imputed with random values drawn from a normal distribution of protein abundances (width=0.2; down-shift=1.8)¹⁹. For sample D01 that had two tumour regions that were prepared separately (low-grade and high-grade tumour regions), maximum protein abundance for each protein was used for differential abundance analyses.

For Tumour vs. NAT comparison of protein-coding RNAs, the data used for the analyses described in this manuscript were obtained from the cBioPortal on July 2020(nₜᵤₘₒᵤᵣ=499, n_{NAT}=53). Mann-Whitney U-test was used for all comparisons, with multiple testing correction using the Benjamini-Hochberg method. Missing values were imputed with random values drawn from a random uniform distribution of RNA transcript per million counts between 0 and 1. All data was log 2-transformed.

### Similarity Between Groups

Euclidean distance was calculated for each tumour and median NAT pair, using protein abundance. Only proteins detected in all samples (n=2,309) were used. IDC/CA groups were determined based on the presence of IDC or CA pathology (IDC/CA+, n=11) or not (IDC/CA−, n=29). Hypoxia groups were determined by median dichotomization (median score²⁰=−1).

### Pathway Enrichment Analysis

Pre-ranked gene set enrichment analysis (GSEA)^{21,22} (v.4.0.3, nₚₑᵣₘᵤₜₐₜᵢₒₙₛ=1000; max size=500; min size=15; enrichment statistic=weighted; normalization mode: meandiv) was performed to identify hallmark gene sets²³ that were enriched in each group. For each comparison (Tumour versus NAT, visible versus invisible tumour, and visible versus invisible NAT), proteins were ranked by log 2 fold change. GSEA was run on each group and adjusted for significance separately but visualized together to better show potential overlaps in hallmark gene sets.

Association Analysis of mpMRl Visibility Hallmarks on RNA and Protein Abundances

To identify protein-coding RNAs associated with mpMRl visibility hallmarks, univariate association tests—Spearman's p for continuous values, Mann-Whitney U test for binary values—were performed with each RNA (log 2-transformed transcripts per million (TPM)) in the discovery cohort⁹ (n=144) against the following mpMRl visibility hallmarks⁵: percent genome altered (PGA), hypoxia (Ragnum score), presence of intraductal carcinoma or cribriform architecture (IDC/CA), and expression of 8 RNAs (SChLAP1, SNORA12, SNORA54, SNORD68, SNORD3A, SNORD33, SNORA37, SCARNA5). RNAs that had <5 TPM in less than 2 samples were excluded from further analysis. Where the calculated p-value was <2×10^{—16}, p-values were imputed based on the effect size. RNAs that were associated with at least one hallmark in the discovery cohort (FDR<0.2) were evaluated in this cohorts (n=40). Hallmark-associated mRNAs that had a corresponding protein detected (n=3,791) in our cohort were carried forward for validation of protein associations with visibility hallmarks (n=40). Proteins were considered validated if they were also significantly associated with the same hallmark at the protein level (FDR<0.2) and had the same directionality as the corresponding RNA association.

Protein Signature to Predict mpMRl-Visible Tumours

We considered proteins detected in all tumour samples (n=2,710) for mpMRl-visibility protein signature development. For sample D01 that had two tumour regions that were prepared separately, the maximum protein abundance for each protein was used. Protein abundances were log 2-transformed and selected for associations with mpMRl-visibility using leave-one-out (n=40) cross validation of Least Absolute Shrinkage and Selection Operator (LASSO) logistic regression(glmnet v4.1), with inner leave-one-out cross validation for lambda selection using the “one-standard-error” rule. In each fold, all 2,710 proteins were initially included as predictors to predict tumour visibility, and the number of times each predictor was selected by the LASSO model were tallied across all folds. Three proteins (LDHB, SRD5A2, GNA11) were consistently chosen as predictors (chosen in at least 15 out of 40 folds) and were used to build a logistic regression model to predict tumour visibility. The performance of the three-protein logistic regression model was assessed using leave-one-out (n=40) cross validation, with AUC and ROC confidence intervals calculated using the pROC package (v1.17.0.1). The three protein signature was tested for synergy in predicting tumour visibility with nimbosus hallmarks and our previously discovered snoRNA signatures using leave-one-out (n=40) cross validation of logistic regression models that included the additional predictors. The nimbosus hallmarks considered were PGA, SCHLAP1, hypoxia and IDC/CA. As described previously, significantly differentially abundant snoRNAs (FDR<0.05) were determined per folds. The three protein logistic regression model (LDHB, SRD5A2, GNA11) was also trained on our full cohort (n=40) and applied to an independent cohort of 76 intermediate-risk prostate cancer samples with log 2-transformed protein abundance of the three proteins⁹. The final logistic regression model had intercept of 274.388, and coefficients of (−)6.439, (−) 1.824 and (−)1.807 for LDHB, SRD5A2, GNA11, respectively. In this independent cohort, samples were dichotomized by logistic regression model predicted mpMRl-visibility probability at the cutoff of 0.5 and the sample groups were tested for differences in biochemical-relapse-free survival using Cox proportional-hazards modeling.

### Example 2

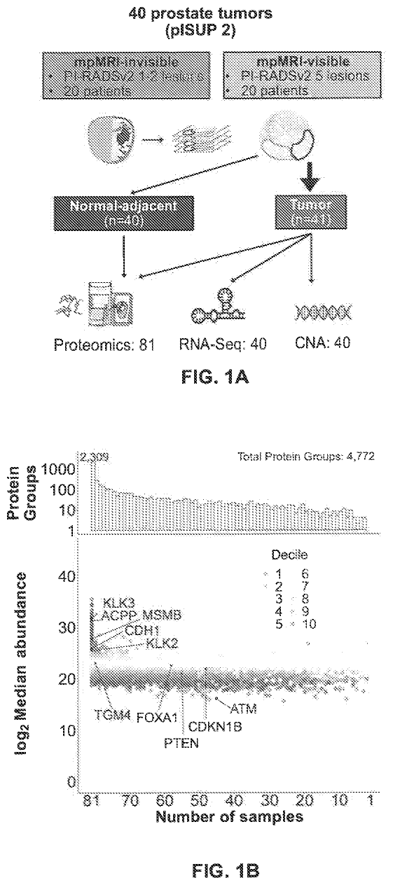

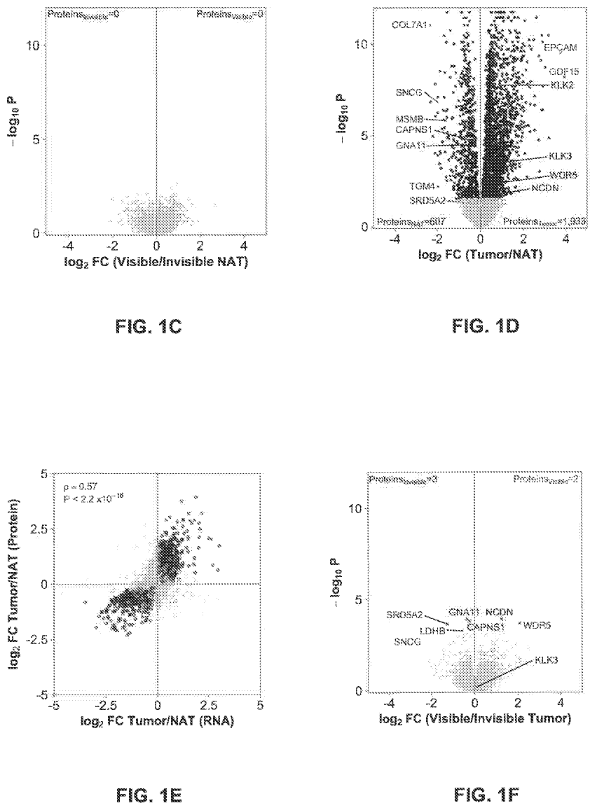

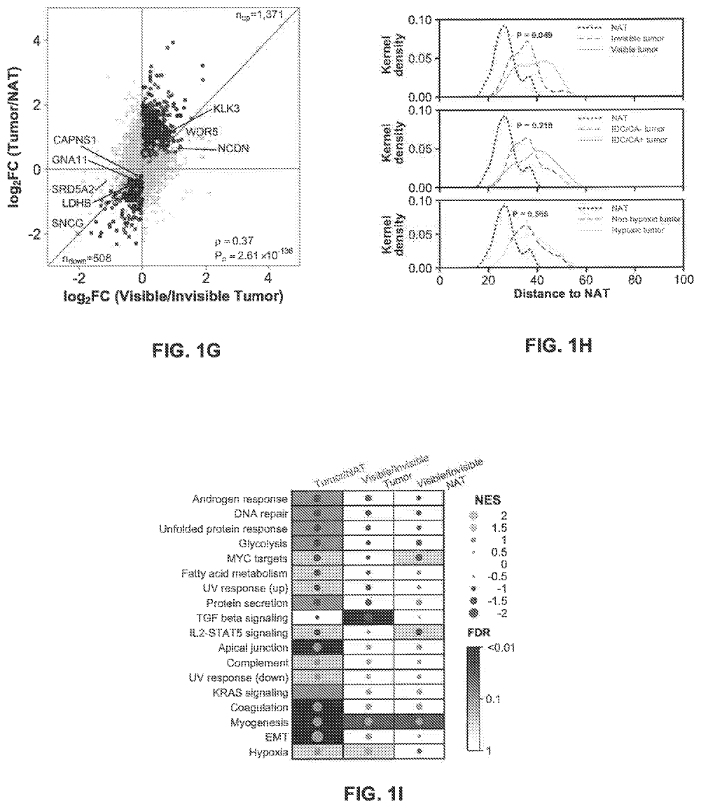

Global proteomic analysis on twenty mpMRl-invisible (PI-RADSv2 1-2) and twenty mpMRI-visible (PI-RADSv2 5) tumours, along with histologically normal tissue adjacent to the tumour (NATs) from all samples was performed using methods as described in Example 1 (also see FIG. 1A; Table 1). All tumours had a solitary pathological ISUP grade group 2 lesion larger than 1.5 cm, and matched copy number and transcriptome profiling⁵. For one patient, Gleason Grade 3 and 4 regions were analyzed separately, yielding a total of 81 proteomes.

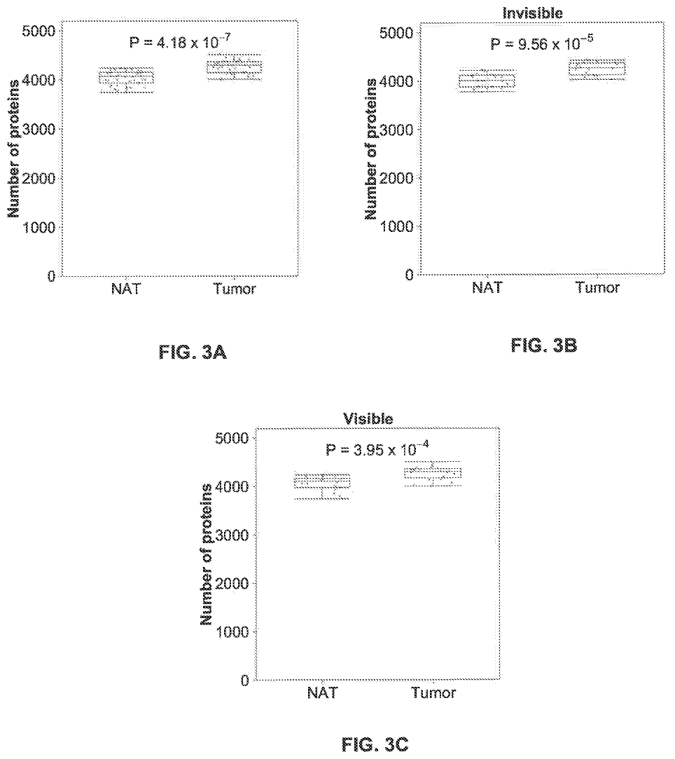

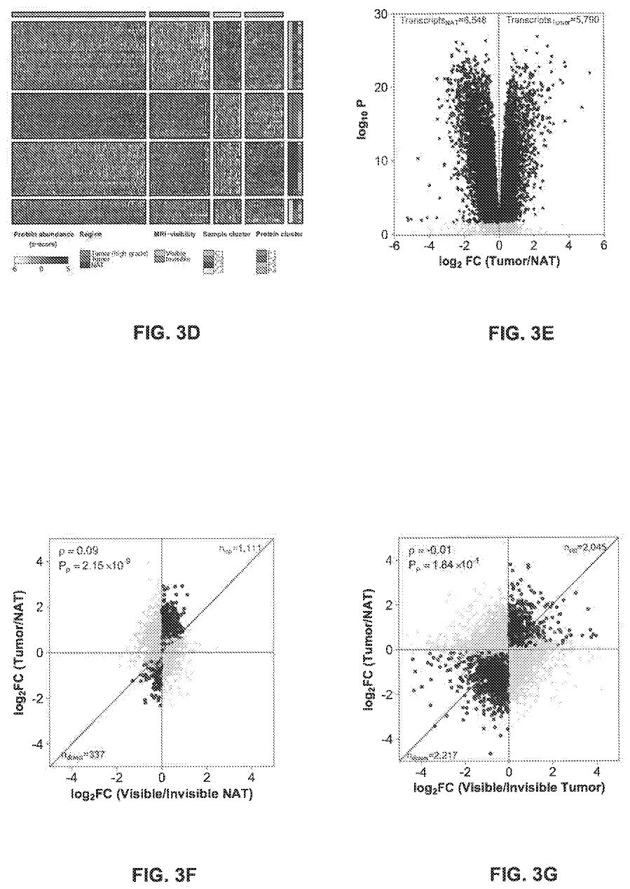

4,772 proteins were quantified of which 2,309 were detected in all 81 samples (FIG. 1B). These universally detected proteins included prostate specific antigen (PSA/KLK3) and prostatic acid phosphatase (ACPP)⁹. There were fewer proteins detected in the NATs compared to tumour samples (n_{NAT}=4,040±138, n_{Tumour}=4,266±132, mean protein number±standard deviation, p=4.18×10⁻⁷; FIG. 3A); this effect was independent of mpMRl-visibility (interaction term p=0.570, FIGS. 3B-3C). Four protein subtypes and four sample subtypes were identified (FIG. 3D). Protein subtype P1 preferentially contained proteins associated with immune response and extracellular matrix organization that were more abundant in tumours than NATs while protein subtype P4 preferentially contained proteins associated with muscle contraction and cellular reorganization that were elevated in NATs (Table 2). Tumours and NATs largely clustered separately (Adjusted Rand Index [ARI]=0.220, p=0.001) but mpMRl-visible and -invisible tumours did not (ARI=−0.008, p=0.641).

### Example 3

It was hypothesized that the tumour microenvironment might influence tumour visibility on mpMRI^{6,7}. The abundance of each protein in NAT from patients with mpMRl-visible tumour was compared to that of mpMRl-invisible tumour using the analysis method as descried in Example 1 tumour. Surprisingly, not a single protein differed (FIG. 1C). By contrast, the expected¹⁰ large differences between the proteomes of tumours and NAT was observed (FIG. 1D). Fully 62% of the detected proteome (2,543/4,314 proteins) was significantly different between tumours and NATs, including known tumour-enriched proteins like EPCAM and KLK3 (FDR<0.05). To verify these proteome data, transcriptome analysis in the TOGA cohort was performed. A similar 61% of the protein-coding transcriptome (12,338/20,233 RNAs) differed significantly between tumours and NATs (FIG. 3E). Most proteome-level tumour-NAT proteomic differences were verified in the transcriptome (Spearman's p=0.57, p<2.2×10⁻¹⁶, FIG. 1E). While there was no difference in mpMRl-visible and -invisible NAT proteomes, a small number of proteins exhibited large differences in tumour tissue. Five proteins were differentially abundant between mpMRl-visible and mpMRl-invisible tumours: SRD5A2, GNA11, CAPNS1, NCDN and WDR5. Four of these were also differentially abundant between tumours and NATs. (FIG. 1F). These results refute the initial hypothesis of a stromal origin to mpMRl visibility.

Given the modest differences between mpMRl-visible and -invisible tumor proteomes, it was hypothesized that mpMRl-invisible tumors might reflect an intermediate state between NATs and mpMRl-visibility. Consistent with this hypothesis, protein abundance differences associated with mpMRl-visibility were correlated with NAT-tumor differences (Spearman's p=0.37; p<1×10⁻¹⁶, FIG. 1G). The protein abundance differences associated with mpMRl visibility was not observed in either the NAT proteomes (Spearman's p=0.09; p=2.15×10⁻⁹, FIG. 3F), nor in the matched tumor transcriptomes (Spearman's p=−0.01; p=0.18, FIG. 3G), the latter highlighting the importance of studying proteins rather than transcripts. The proteome of mpMRl-invisible tumours was more similar to that of NATs than were mpMRl-visible tumours (p=0.049; FIG. 1H), likely contributing to their invisibility¹¹. Similar but weaker trends were seen for hypoxic tumours and for tumours with IDC/CA histology. Altered pathways in mpMRl-visible tumours vs mpMRl-invisible tumours overlapped substantially with those distinguishing tumours from NATs (FIG. 11). Notably, EMT and myogenesis genes were enriched in mpMRl-invisible tumours, consistent with reports that stromal and extracellular matrix gene abundances were enriched in mpMRl-invisible tumours^{11,12}. mpMRl-visible tumours were enriched in pathways associated with advanced disease, including androgen response, DNA repair, and MYC and TGFβ signaling^{13,14}. These finding further explain the aggressive clinical behaviour of mpMRl-visible tumours, concordant with their increased PTEN loss¹², higher Oncotype and Decipher genomic classifier scores^{16,16}, and elevated nimbosus hallmarks⁵.

### Example 4

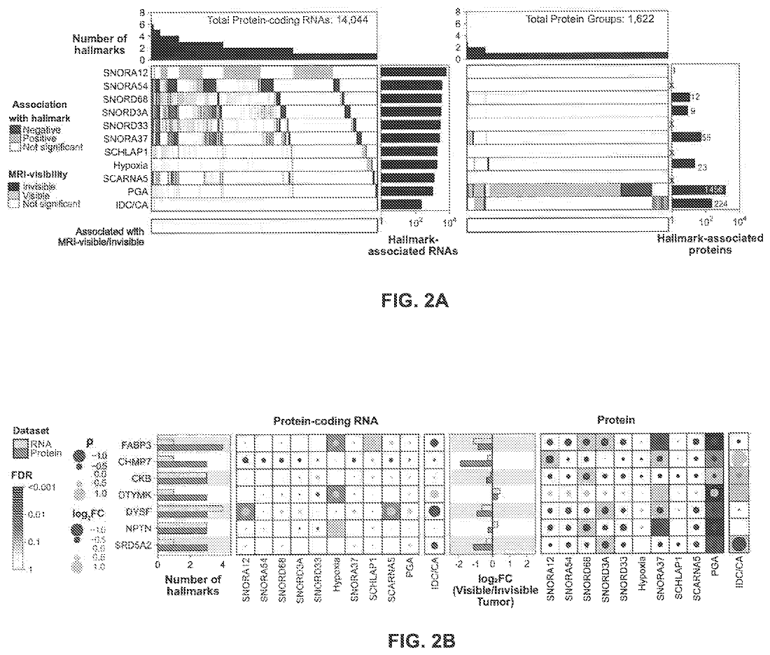

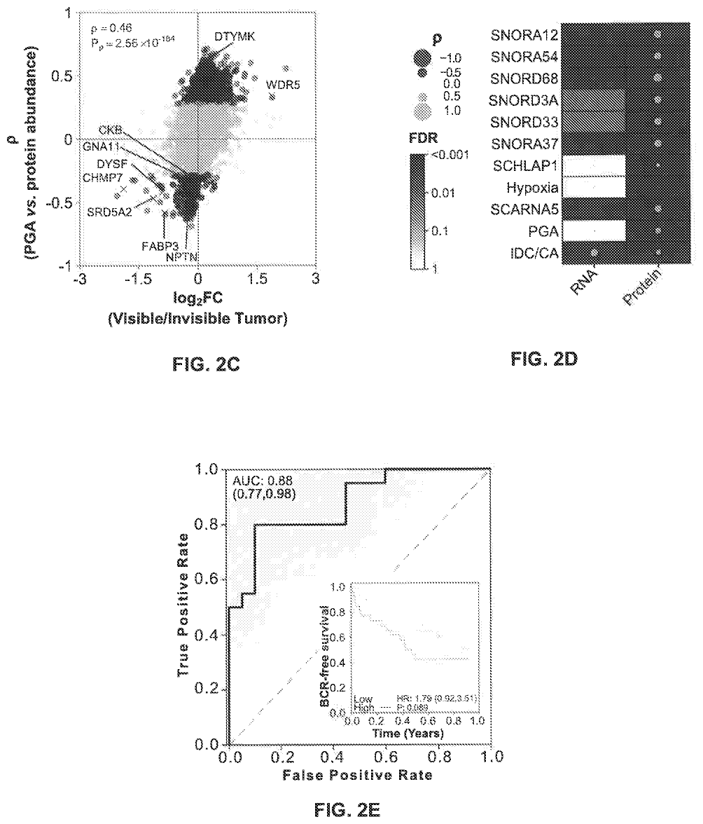

To identify protein-coding RNAs and proteins associated with mpMRl-visibility and disease aggression, the nimbosus hallmarks were focused on^{6,6}. An independent discovery cohort of 144 NCCN intermediate-risk tumours was used to discover associations between RNA abundance and each hallmark as described in Example 1 ^{9,17}. Significant transcriptome associations were then validated in this cohort at the RNA levels, and confirmed in the proteome. 14,044 protein-coding RNAs and 1,622 proteins associated with at least one nimbosus hallmark were identified (FIG. 2A). PGA and IDC/CA showed the largest effects on the transcriptome and proteome. Three proteins were associated with mpMRl-visibility, PGA and IDC/CA: SRD5A2, GNA11 and WDR5. Proteins that were more abundant in mpMRl-invisible tumours were also negatively correlated with hallmarks (FIG. 2B). Proteins that were associated with high PGA were also preferentially associated with mpMRl-visibility (Spearman's p=0.43, p=3.29×10⁻¹⁵¹; FIG. 2C). mpMRl-visibility was also strongly associated with aggressive hallmarks such as hypoxia, presence of IDC/CA, and SChLAP1 expression through proteins, rather than protein-coding RNAs (FIG. 2D).

### Example 5

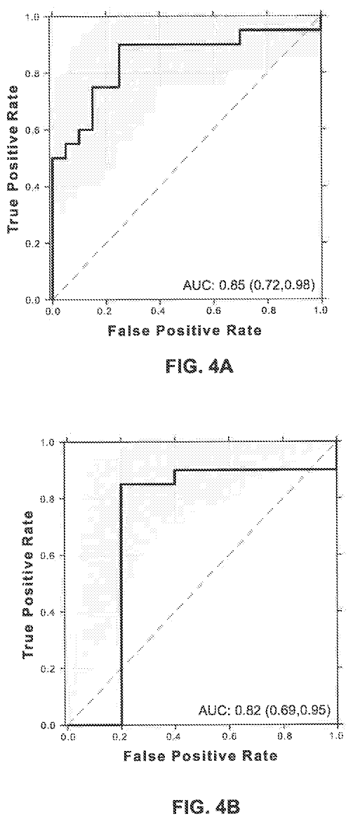

To identify a molecular biomarker that predicts mpMRl-visibility, statistical machine-learning was applied to the dataset as described in Example 1. This created a three-protein biomarker (LDHB, GNA11, SRD5A2) that could predict mpMRl-visibility with an AUC of 0.88 (0195%=0.77-0.98, FIG. 2E). This biomarker was associated with worse biochemical recurrence-free survival in an independent cohort of 76 predominantly NCCN intermediate-risk tumours (HR=1.79; 0195%=0.92-3.51; p=0.089; median follow-up 6.02 years)⁹. The performance of the 3-protein classifier was independent of the nimbosus hallmarks (AUC 0.85, 0195%=0.72-0.98; FIG. 4A) and snoRNAs⁵ (AUC 0.82, 0195%=0.69-0.95; FIG. 4B).

### Example 6

The ability for CAPNS1, GNA11, SRD5A2, LDHB, WDR5, NCDN each individually and in pairs to predict mpMRl visibility was assessed in the dataset as described in Example 1 (see Table 3 and Table 4).

### Example 7

The methods described herein can be used to predict mpMRl visibility in treatment-naïve prostate cancer patients, Biopsy tissues can be obtained by transrectal biopsy or transperineal biopsy, and tissue cores containing the tumour would be used for mass spectrometry. The entire tissue core can be cryopulverized, and proteins can be extracted for protein digestion and mass spectrometry analysis as described in Example 1.

TABLE 1
Summary and characteristics of patient cohort

| | | Serum | PSA | Pathologic | | Pathological | Radiologic |
|---|---|---|---|---|---|---|---|
| Patient | | PSA | density | Gleason | Area | prostate volume | prostate volume |
| ID | Age | (ng/mL) | (ng/mL\hat{ }2) | score | (mm\hat{ }2) | (mL) | ml |
| A01 | 61 | 5.1 | 0.06 | 3 + 4 | 40.288 | 80 | 74 |
| A02 | 68 | 6.7 | 0.18 | 3 + 4 | 134.229 | 37 | 41 |
| A03 | 53 | 3.9 | 0.19 | 3 + 4 | 59.824 | 20.1 | 16.6 |
| A04 | 66 | 7.3 | 0.13 | 3 + 4 | 80.975 | 55 | 63 |
| A05 | 53 | 4.3 | 0.1 | 3 + 4 | 115.208 | 41 | 38 |
| A06 | 51 | 3.1 | 0.08 | 3 + 4 | 118.148 | 38.9 | 37 |
| A07 | 75 | 9.3 | 0.11 | 3 + 4 | 50.851 | 88 | 84 |
| A08 | 60 | 37.8 | 1.02 | 3 + 4 | 373.766 | 37 | 30 |
| A09 | 70 | 7 | 0.13 | 3 + 4 | 86.469 | 56 | 58 |
| A10 | 65 | 5 | 0.12 | 3 + 4 | 114.171 | 40.9 | 40 |
| A11 | 51 | 6.3 | 0.23 | 3 + 4 | 82.212 | 28 | 26 |
| A12 | 65 | 8.6 | 0.18 | 3 + 4 | 521.538 | 47 | 35 |
| A13 | 55 | 7.5 | 0.2 | 3 + 4 | 154.64 | 37 | 28 |
| A14 | 60 | 2.7 | 0.05 | 3 + 4 | 72.812 | 50 | 49 |
| A15 | 47 | 6.9 | 0.25 | 3 + 4 | 62.672 | 28 | 30 |
| A16 | 47 | 6.7 | 0.16 | 3 + 4 | 93.415 | 43 | 36 |
| A17 | 68 | 2.1 | 0.07 | 3 + 4 | 102.895 | 30 | 33 |
| A18 | 60 | 4.6 | 0.09 | 3 + 4 | 211.122 | 51 | 50 |
| A19 | 63 | 4.8 | 0.17 | 3 + 4 | 139.321 | 29 | 25 |
| A20 | 67 | 11 | 0.15 | 3 + 4 | NA | 75 | 52 |
| D01 | 72 | 5.24 | 0.1 | 3 + 4 | 232.885 | 50 | 43 |
| D01 | 72 | 5.24 | 0.1 | 3 + 4 | 29.393 | 50 | 43 |
| D02 | 64 | 5.6 | 0.11 | 3 + 4 | 119.565 | 51.3 | 34 |
| D03 | 65 | 2.6 | 0.07 | 3 + 4 | 168.251 | 35.1 | 34 |
| D04 | 74 | 14 | 0.14 | 3 + 4 | 267.271 | 97.5 | 106 |
| D05 | 75 | 5 | 0.11 | 3 + 4 | 277.838 | 47.5 | 38 |
| D06 | 64 | 4.9 | 0.08 | 3 + 4 | 139.216 | 59.8 | 37 |
| D07 | 69 | 19.4 | 0.29 | 3 + 4 | 222.246 | 67 | 68 |
| D08 | 71 | 3.8 | 0.15 | 3 + 4 | 157.285 | 26 | 25 |
| D09 | 71 | 9.7 | 0.21 | 3 + 4 | 282.88 | 47 | 46 |
| D10 | 55 | 9.7 | 0.26 | 3 + 4 | 287.5 | 38 | 32 |
| D11 | 65 | 8 | 0.16 | 3 + 4 | 170.54 | 49 | 42 |
| D12 | 52 | 8.6 | 0.34 | 3 + 4 | 117.2045 | 25 | 17 |
| D13 | 52 | 3.4 | 0.09 | 3 + 4 | 124.2577 | 40 | 39 |
| D14 | 60 | 5.6 | 0.22 | 3 + 4 | 47.57729 | 26 | 27 |
| D15 | 60 | 8.6 | 0.25 | 3 + 4 | 97.46566 | 34 | 35 |
| D16 | 64 | 6.2 | 0.08 | 3 + 4 | 71.87653 | 80 | 68 |
| D17 | 54 | 3.6 | 0.12 | 3 + 4 | 128.3914 | 30 | 28 |
| D18 | 49 | 7 | 0.26 | 3 + 4 | 95.77413 | 27 | 23 |
| D19 | 56 | 12.5 | 0.29 | 3 + 4 | 360.7119 | 43 | 40 |
| D20 | 61 | 13.9 | 0.27 | 3 + 4 | 462.8947 | 52 | 42 |

| | | Pathologic | | | | |
|---|---|---|---|---|---|---|
| | Tumor | tumor | | | Percent | Percent |
| Patient | size | volume | Tumor | T | cribriform | intraductal |
| ID | (cm) | (ml) | location | category | architecture | architecture |
| A01 | 2.6 | 4.8 | PZ | pT2c | 0 | 0 |
| A02 | 2.9 | 8 | PZ | pT2c | 0 | 0 |
| A03 | 2 | 2 | PZ | pT3a | 0 | 0 |
| A04 | 1.9 | 2.5 | TZ | pT2c | 0 | 0 |
| A05 | 1.9 | 2.9 | PZ | pT3a | 0 | 0 |
| A06 | 1.5 | 2 | TZ | pT2c | 0 | 0 |
| A07 | 2.4 | 4.4 | PZ | pT3a | 0 | 0 |
| A08 | 3.3 | 11 | TZ | pT2c | <5 | 0 |
| A09 | 1.5 | 2.8 | TZ | pT2c | <5 | 0 |
| A10 | 2.3 | 2 | PZ | pT2c | 0 | 0 |
| A11 | 1.8 | 4.2 | TZ | pT2c | 0 | 0 |
| A12 | 4.2 | 20 | TZ | pT2c | 0 | 0 |
| A13 | 1.8 | 4.5 | PZ | pT2a | <5 | 0 |
| A14 | 1.8 | 7.5 | PZ | pT2a | 0 | 0 |
| A15 | 2.3 | 2.3 | PZ | pT2a | 5 | 0 |
| A16 | 2.1 | 3.4 | PZ | pT2c | 0 | 0 |
| A17 | 1.7 | 4.5 | PZ | pT2c | 0 | 0 |
| A18 | 2.8 | 10.2 | PZ | pT2b | 5 | 0 |
| A19 | 2 | 2 | TZ | pT2c | 0 | 0 |
| A20 | 4.3 | 10 | TZ | pT3a | <5 | 0 |
| D01 | 2.1 | 7.5 | TZ | pT2b | 0 | 0 |
| D01 | 2.1 | 7.5 | TZ | pT2b | 0 | 0 |
| D02 | 1.8 | 5.1 | PZ | pT3a | 0 | 0 |
| D03 | 3 | 5 | PZ | pT3a | 30 | 20 |
| D04 | 3.2 | 10 | TZ | pT2c | 0 | 0 |
| D05 | 3.8 | 14.3 | PZ | pT3a | 0 | <5 |
| D06 | 3.3 | 12 | PZ | pT2c | 5 | 0 |
| D07 | 2.6 | 3.4 | TZ | pT3a | 0 | 0 |
| D08 | 1.9 | 3.9 | PZ | pT3a | 10 | 0 |
| D09 | 2.9 | 7 | TZ | pT2c | 0 | 0 |
| D10 | 2.9 | 5 | TZ | pT2c | 20 | 0 |
| D11 | 1.9 | 15 | PZ | pT3a | 10 | 0 |
| D12 | 1.8 | 3 | PZ | pT2b | 5 | 0 |
| D13 | 2.1 | 4 | PZ | pT2b | 0 | 0 |
| D14 | 1.8 | 3.1 | PZ | pT3a | <5 | 0 |
| D15 | 3.3 | 10.2 | PZ | pT3b | 0 | 0 |
| D16 | 2.9 | 8 | PZ | pT3b | 5 | 0 |
| D17 | 2 | 6 | TZ | pT3a | 5 | 0 |
| D18 | 2.4 | 5.4 | PZ | pT2b | 10 | 5 |
| D19 | 3.4 | 8.5 | TZ | pT2c | 0 | 0 |
| D20 | 3.9 | 18 | TZ | pT3a | 0 | 0 |

| Patient | Estimated | Prospective | Retrospective | | | |
|---|---|---|---|---|---|---|
| ID | cellularity | PIRADS v2 | PIRADS v2 | Schlap1 | PGA | Hypoxia |
| A01 | 1 | <3 | <3 | 0 | 0.729999 | −4 |
| A02 | 1 | <3 | 3 | 0 | 0.025689 | −12 |
| A03 | 0.87 | <3 | <3 | 8.2 | 0.172625 | −10 |
| A04 | 0.33 | <3 | <3 | 0 | 0.279235 | −10 |
| A05 | 0.43 | <3 | 3 | 0.69 | 3.600954 | 4 |
| A06 | 1 | <3 | 3 | 0 | 0.08085 | −12 |
| A07 | 0.54 | <3 | <3 | 1.77 | 3.836397 | 6 |
| A08 | 0.71 | <3 | <3 | 0 | 1.101763 | 12 |
| A09 | 1 | <3 | <3 | 5.05 | 0.084757 | −14 |
| A10 | 0.36 | <3 | <3 | 0 | 0.732656 | −10 |
| A11 | 1 | <3 | <3 | 0.17 | 0.075699 | 6 |
| A12 | 1 | <3 | 3 | 0 | 0.359164 | −10 |
| A13 | 0.65 | <3 | 4 | 0.17 | 2.27863 | −2 |
| A14 | 1 | <3 | 3 | 0.3 | 0.410807 | 4 |
| A15 | 0.49 | <3 | <3 | 1.5 | 6.150697 | 4 |
| A16 | 0.28 | <3 | <3 | 0 | 2.245137 | 2 |
| A17 | 0.62 | <3 | <3 | 1.76 | 3.532741 | −2 |
| A18 | 1 | <3 | <3 | 2.87 | 2.031318 | 4 |
| A19 | 1 | <3 | <3 | 0.59 | 0.046095 | −6 |
| A20 | 1 | <3 | <3 | 0 | 1.006187 | −6 |
| D01 | 0.69 | 5 | NA | 5.92 | 5.128115 | −14 |
| D01 | 0.69 | 5 | NA | 5.92 | 5.128115 | −14 |
| D02 | 0.9 | 5 | NA | 1.69 | 0.065201 | 4 |
| D03 | 0.71 | 5 | NA | 96.45 | 1.56454 | 0 |
| D04 | 0.63 | 5 | NA | 0 | 3.526238 | 2 |
| D05 | 0.55 | 5 | NA | 39.21 | 6.144717 | 12 |
| D06 | 0.53 | 5 | NA | 30.22 | 4.221884 | 8 |
| D07 | 0.45 | 5 | NA | 2.94 | 11.14853 | 2 |
| D08 | 1 | 5 | NA | 7.25 | 0.216192 | −4 |
| D09 | 0.81 | 5 | NA | 0.1 | 1.57201 | −20 |
| D10 | 0.71 | 5 | NA | 2.18 | 7.090867 | −4 |
| D11 | 0.58 | 5 | NA | 0.39 | 7.935915 | 8 |
| D12 | 0.42 | 5 | NA | 0.29 | 4.8683 | 2 |
| D13 | 1 | 5 | NA | 5.07 | 0.003659 | −4 |
| D14 | 1 | 5 | NA | 12.14 | 0.490827 | 12 |
| D15 | 1 | 5 | NA | 3.61 | 0.039538 | 12 |
| D16 | 0.54 | 5 | NA | 0.35 | 3.031382 | 8 |
| D17 | 0.7 | 5 | NA | 1.42 | 3.408756 | −2 |
| D18 | 1 | 5 | NA | 25.96 | 0.063857 | 18 |
| D19 | 0.69 | 5 | NA | 0.68 | 4.46311 | −10 |
| D20 | 0.55 | 5 | NA | 0 | 3.691585 | −30 |

| Patient | | | | | | | |
|---|---|---|---|---|---|---|---|
| ID | Snord33 | Snord68 | Snora54 | Snora37 | Snora12 | Scarna5 | Snord3a |
| A01 | 164.79 | 0 | 0 | 0 | 0 | 117.81 | 1043.54 |
| A02 | 124.26 | 0 | 87.02 | 0 | 0 | 31.36 | 1703.68 |
| A03 | 285.29 | 0 | 8.48 | 0 | 0 | 116.64 | 353.69 |
| A04 | 249.64 | 2.96 | 54.43 | 34.63 | 0 | 109.25 | 1690.93 |
| A05 | 164.05 | 4.05 | 19.3 | 17.86 | 34.06 | 120.11 | 664.19 |
| A06 | 223.25 | 14.96 | 20.71 | 10.95 | 49.24 | 60.1 | 936.65 |
| A07 | 623.21 | 136.73 | 46.06 | 30.45 | 17.42 | 119.65 | 730.96 |
| A08 | 458.12 | 29.13 | 55.57 | 79.28 | 12.26 | 87.22 | 944.35 |
| A09 | 293.67 | 0 | 0 | 8.6 | 0 | 115.73 | 2439.96 |
| A10 | 191.66 | 32.34 | 24.36 | 28.98 | 10.53 | 167.55 | 2913.82 |
| A11 | 202.26 | 4.75 | 15.99 | 14.8 | 33.88 | 132.05 | 1485.92 |
| A12 | 185.88 | 0 | 29.54 | 0 | 0 | 118.78 | 1633.79 |
| A13 | 190.73 | 0 | 22.7 | 7.88 | 15.03 | 157.09 | 1072.38 |
| A14 | 148.44 | 0 | 14.1 | 7.83 | 27.74 | 122.87 | 832.54 |
| A15 | 131.72 | 15.07 | 54.41 | 34.62 | 30.87 | 98.03 | 1008.47 |
| A16 | 68.95 | 8.93 | 13.55 | 9.41 | 23.07 | 119.87 | 822.49 |
| A17 | 110.7 | 0.29 | 24.5 | 10.08 | 12.35 | 180.98 | 1631.96 |
| A18 | 89.25 | 0 | 20.46 | 37.87 | 4.42 | 91.91 | 1101.48 |
| A19 | 169.48 | 7.04 | 24.74 | 33.3 | 28.92 | 218.75 | 1923.66 |
| A20 | 311.24 | 27.26 | 85.99 | 35.94 | 23.08 | 164.47 | 2502.69 |
| D01 | 1067.73 | 180.41 | 143.08 | 84.27 | 63.96 | 239.7 | 6979.16 |
| D01 | 1067.73 | 180.41 | 143.08 | 84.27 | 63.96 | 239.7 | 6979.16 |
| D02 | 399.06 | 28.64 | 68.49 | 15.09 | 4.93 | 151.69 | 1442.53 |
| D03 | 583 | 110.07 | 63.1 | 58.4 | 33.69 | 209.14 | 2141.77 |
| D04 | 580.09 | 165.86 | 100.8 | 64.79 | 46.6 | 220.21 | 3730.25 |
| D05 | 492.21 | 48.88 | 61.03 | 48.78 | 2.1 | 89.07 | 1173.24 |
| D06 | 255.51 | 15.89 | 29.82 | 22.08 | 20.3 | 234.39 | 1583.34 |
| D07 | 623.99 | 61.96 | 71.81 | 125.54 | 98.59 | 279.69 | 4397.3 |
| D08 | 631.36 | 134.44 | 47.15 | 31.17 | 25.48 | 253.77 | 2566.46 |
| D09 | 579.67 | 40.82 | 42.81 | 79.25 | 142.79 | 384.38 | 1460.86 |
| D10 | 693.03 | 32.19 | 83.25 | 16.66 | 27.23 | 311.72 | 1946.74 |
| D11 | 163.81 | 55.26 | 40.42 | 37.41 | 37.13 | 203.33 | 2219.72 |
| D12 | 168.84 | 12.19 | 26.71 | 9.89 | 30.3 | 116.36 | 1118.22 |
| D13 | 301.4 | 89.51 | 67.74 | 62.69 | 41.92 | 216.84 | 1446.77 |
| D14 | 265.83 | 13.66 | 29.62 | 35.63 | 42.57 | 209.55 | 1555.83 |
| D15 | 399.92 | 58.09 | 49.81 | 37.46 | 35.32 | 154.01 | 1644.04 |
| D16 | 284.55 | 17.07 | 42.21 | 15.03 | 17.19 | 109.55 | 2006.42 |
| D17 | 167.37 | 28.94 | 34.57 | 31.99 | 32.18 | 154.17 | 1739.64 |
| D18 | 412.78 | 62.52 | 47.92 | 9.85 | 48.33 | 205.78 | 2165.89 |
| D19 | 526.82 | 61.64 | 56.71 | 36.94 | 68.32 | 246.35 | 3415.73 |
| D20 | 1659.19 | 74.29 | 283.33 | 148.76 | 272.01 | 472.95 | 4406.68 |

TABLE 2
Pathway analysis of protein clusters

| | | term | intersection | | | | | |
|---|---|---|---|---|---|---|---|---|
| subtype | p-value | size | size | precision | recall | term id | source | term name |
| P4 | 3.10E−12 | 39 | 11 | 0.087302 | 0.282051 | GO:0030049 | GO:BP | muscle filament sliding |
| P4 | 3.10E−12 | 39 | 11 | 0.087302 | 0.282051 | GO:0033275 | GO:BP | actin-myosin filament sliding |
| P4 | 1.46E−09 | 363 | 20 | 0.15873 | 0.055096 | GO:0006936 | GO:BP | muscle contraction |
| P4 | 2.56E−09 | 471 | 22 | 0.174603 | 0.046709 | GO:0003012 | GO:BP | muscle system process |
| P4 | 1.60E−06 | 122 | 11 | 0.087302 | 0.090164 | GO:0070252 | GO:BP | actin-mediated cell contraction |
| P4 | 1.54E−05 | 151 | 11 | 0.087302 | 0.072848 | GO:0030048 | GO:BP | actin filament-based movement |
| P4 | 2.27E−05 | 63 | 8 | 0.063492 | 0.126984 | GO:0030239 | GO:BP | myofibril assembly |
| P4 | 2.58E−05 | 64 | 8 | 0.063492 | 0.125 | GO:0055002 | GO:BP | striated muscle cell development |
| P4 | 2.71E−05 | 42 | 7 | 0.055556 | 0.166667 | GO:0045214 | GO:BP | sarcomere organization |
| P4 | 0.000327 | 810 | 21 | 0.166667 | 0.025926 | GO:0030029 | GO:BP | actin filament-based process |
| P4 | 0.00074 | 175 | 10 | 0.079365 | 0.057143 | GO:0055001 | GO:BP | muscle cell development |
| P4 | 0.001591 | 108 | 8 | 0.063492 | 0.074074 | GO:0010927 | GO:BP | cellular component assembly involved in |
| | | | | | | | | morphogenesis |
| P4 | 0.002403 | 199 | 10 | 0.079365 | 0.050251 | GO:0031032 | GO:BP | actomyosin structure organization |
| P4 | 0.008663 | 180 | 9 | 0.071429 | 0.05 | GO:0006941 | GO:BP | striated muscle contraction |
| P4 | 0.009109 | 285 | 11 | 0.087302 | 0.038596 | GO:0051146 | GO:BP | striated muscle cell differentiation |
| P3 | 0.00022 | 3519 | 84 | 0.333333 | 0.02387 | GO:0051641 | GO:BP | cellular localization |
| P3 | 0.001186 | 2778 | 69 | 0.27381 | 0.024838 | GO:0051649 | GO:BP | establishment of localization in cell |
| P3 | 0.005535 | 3 | 3 | 0.011905 | 1 | GO:0002484 | GO:BP | antigen processing and presentation of endogenous |
| | | | | | | | | peptide antigen via MHC class I via ER pathway |
| P3 | 0.005535 | 3 | 3 | 0.011905 | 1 | GO:0002486 | GO:BP | antigen processing and presentation of endogenous |
| | | | | | | | | peptide antigen via MHC class I via ER pathway, TAP- |
| | | | | | | | | independent |
| P3 | 0.018038 | 2193 | 55 | 0.218254 | 0.02508 | GO:0016192 | GO:BP | vesicle-mediated transport |
| P3 | 0.028716 | 1842 | 48 | 0.190476 | 0.026059 | GO:0034613 | GO:BP | cellular protein localization |
| P3 | 0.035024 | 1856 | 48 | 0.190476 | 0.025862 | GO:0070727 | GO:BP | cellular macromolecule localization |
| P2 | 7.54E−19 | 87 | 28 | 0.053846 | 0.321839 | GO:0070125 | GO:BP | mitochondrial translational elongation |
| P2 | 2.64E−17 | 135 | 32 | 0.061538 | 0.237037 | GO:0032543 | GO:BP | mitochondrial translation |
| P2 | 2.74E−16 | 88 | 26 | 0.05 | 0.295455 | GO:0070126 | GO:BP | mitochondrial translational termination |
| P2 | 1.73E−14 | 143 | 30 | 0.057692 | 0.20979 | GO:0006414 | GO:BP | translational elongation |
| P2 | 1.92E−14 | 166 | 32 | 0.061538 | 0.192771 | GO:0140053 | GO:BP | mitochondrial gene expression |
| P2 | 2.76E−14 | 104 | 26 | 0.05 | 0.25 | GO:0006415 | GO:BP | translational termination |
| P2 | 7.74E−09 | 228 | 30 | 0.057692 | 0.131579 | GO:0043624 | GO:BP | cellular protein complex disassembly |
| P2 | 1.16E−07 | 337 | 35 | 0.067308 | 0.103858 | GO:0032984 | GO:BP | protein-containing complex disassembly |
| P2 | 1.81E−07 | 1284 | 79 | 0.151923 | 0.061526 | GO:0043603 | GO:BP | cellular amide metabolic process |
| P2 | 6.25E−07 | 804 | 57 | 0.109615 | 0.070896 | GO:0006412 | GO:BP | translation |
| P2 | 1.10E−06 | 975 | 64 | 0.123077 | 0.065641 | GO:0043604 | GO:BP | amide biosynthetic process |
| P2 | 2.46E−06 | 834 | 57 | 0.109615 | 0.068345 | GO:0043043 | GO:BP | peptide biosynthetic process |
| P2 | 1.76E−05 | 998 | 62 | 0.119231 | 0.062124 | GO:0006518 | GO:BP | peptide metabolic process |
| P2 | 3.00E−05 | 2017 | 101 | 0.194231 | 0.050074 | GO:0043933 | GO:BP | protein-containing complex subunit organization |
| P2 | 7.33E−05 | 1889 | 95 | 0.182692 | 0.050291 | GO:1901566 | GO:BP | organonitrogen compound biosynthetic process |
| P2 | 0.000156 | 1010 | 60 | 0.115385 | 0.059406 | GO:0006396 | GO:BP | RNA processing |
| P2 | 0.000599 | 577 | 40 | 0.076923 | 0.069324 | GO:0022411 | GO:BP | cellular component disassembly |
| P2 | 0.00126 | 11915 | 393 | 0.755769 | 0.032984 | GO:0008152 | GO:BP | metabolic process |
| P2 | 0.002064 | 10941 | 366 | 0.703846 | 0.033452 | GO:0044237 | GO:BP | cellular metabolic process |
| P2 | 0.003695 | 6904 | 250 | 0.480769 | 0.036211 | GO:1901564 | GO:BP | organonitrogen compound metabolic process |
| P2 | 0.005968 | 11397 | 376 | 0.723077 | 0.032991 | GO:0071704 | GO:BP | organic substance metabolic process |
| P2 | 0.011007 | 532 | 35 | 0.067308 | 0.065789 | GO:0006397 | GO:BP | mRNA processing |
| P2 | 0.017374 | 6391 | 231 | 0.444231 | 0.036145 | GO:0009058 | GO:BP | biosynthetic process |
| P2 | 0.02019 | 3148 | 129 | 0.248077 | 0.040978 | GO:0033036 | GO:BP | macromolecule localization |
| P2 | 0.021783 | 6210 | 225 | 0.432692 | 0.036232 | GO:0044249 | GO:BP | cellular biosynthetic process |
| P2 | 0.025317 | 1924 | 87 | 0.167308 | 0.045218 | GO:0044281 | GO:BP | small molecule metabolic process |
| P2 | 0.028006 | 6297 | 227 | 0.436538 | 0.036049 | GO:1901576 | GO:BP | organic substance biosynthetic process |
| P2 | 0.031228 | 535 | 34 | 0.065385 | 0.063551 | GO:0034660 | GO:BP | ncRNA metabolic process |
| P2 | 0.041036 | 41 | 8 | 0.015385 | 0.195122 | GO:0046782 | GO:BP | regulation of viral transcription |
| P2 | 0.045792 | 2657 | 111 | 0.213462 | 0.041776 | GO:0071702 | GO:BP | organic substance transport |
| P2 | 0.047019 | 2216 | 96 | 0.184615 | 0.043321 | GO:0071705 | GO:BP | nitrogen compound transport |
| P1 | 1.37E−10 | 144 | 17 | 0.094972 | 0.118056 | GO:0019730 | GO:BP | antimicrobial humoral response |
| P1 | 1.78E−10 | 422 | 26 | 0.145251 | 0.061611 | GO:0045229 | GO:BP | external encapsulating structure organization |
| P1 | 1.14E−09 | 419 | 25 | 0.139665 | 0.059666 | GO:0030198 | GO:BP | extracellular matrix organization |
| P1 | 1.21E−09 | 420 | 25 | 0.139665 | 0.059524 | GO:0043062 | GO:BP | extracellular structure organization |
| P1 | 9.18E−09 | 383 | 23 | 0.128492 | 0.060052 | GO:0006959 | GO:BP | humoral immune response |
| P1 | 1.07E−08 | 349 | 22 | 0.122905 | 0.063037 | GO:0042742 | GO:BP | defense response to bacterium |
| P1 | 1.60E−07 | 483 | 24 | 0.134078 | 0.049689 | GO:0043312 | GO:BP | neutrophil degranulation |
| P1 | 1.98E−07 | 488 | 24 | 0.134078 | 0.04918 | GO:0002283 | GO:BP | neutrophil activation involved in immune response |
| P1 | 3.25E−07 | 500 | 24 | 0.134078 | 0.048 | GO:0002446 | GO:BP | neutrophil mediated immunity |
| P1 | 3.53E−07 | 502 | 24 | 0.134078 | 0.047809 | GO:0042119 | GO:BP | neutrophil activation |
| P1 | 4.67E−07 | 509 | 24 | 0.134078 | 0.047151 | GO:0036230 | GO:BP | granulocyte activation |
| P1 | 8.35E−07 | 61 | 10 | 0.055866 | 0.163934 | GO:0019731 | GO:BP | antibacterial humoral response |
| P1 | 1.43E−06 | 538 | 24 | 0.134078 | 0.04461 | GO:0043299 | GO:BP | leukocyte degranulation |
| P1 | 2.38E−06 | 552 | 24 | 0.134078 | 0.043478 | GO:0002275 | GO:BP | myeloid cell activation involved in immune response |
| P1 | 2.95E−06 | 558 | 24 | 0.134078 | 0.043011 | GO:0002444 | GO:BP | myeloid leukocyte mediated immunity |
| P1 | 4.88E−06 | 437 | 21 | 0.117318 | 0.048055 | GO:0052548 | GO:BP | regulation of endopeptidase activity |
| P1 | 6.67E−06 | 888 | 30 | 0.167598 | 0.033784 | GO:0002443 | GO:BP | leukocyte mediated immunity |
| P1 | 1.57E−05 | 467 | 21 | 0.117318 | 0.044968 | GO:0052547 | GO:BP | regulation of peptidase activity |
| P1 | 2.37E−05 | 671 | 25 | 0.139665 | 0.037258 | GO:0002274 | GO:BP | myeloid leukocyte activation |
| P1 | 3.97E−05 | 1135 | 33 | 0.184358 | 0.029075 | GO:0002252 | GO:BP | immune effector process |
| P1 | 4.20E−05 | 743 | 26 | 0.145251 | 0.034993 | GO:0009617 | GO:BP | response to bacterium |
| P1 | 4.49E−05 | 2193 | 49 | 0.273743 | 0.022344 | GO:0016192 | GO:BP | vesicle-mediated transport |
| P1 | 0.000143 | 790 | 26 | 0.145251 | 0.032911 | GO:0045055 | GO:BP | regulated exocytosis |
| P1 | 0.000158 | 906 | 28 | 0.156425 | 0.030905 | GO:0006887 | GO:BP | exocytosis |
| P1 | 0.000216 | 81 | 9 | 0.050279 | 0.111111 | GO:0061844 | GO:BP | antimicrobial humoral immune response mediated by |
| | | | | | | | | antimicrobial peptide |
| P1 | 0.000218 | 863 | 27 | 0.150838 | 0.031286 | GO:0097435 | GO:BP | supramolecular fiber organization |
| P1 | 0.000412 | 723 | 24 | 0.134078 | 0.033195 | GO:0002366 | GO:BP | leukocyte activation involved in immune response |
| P1 | 0.000452 | 252 | 14 | 0.078212 | 0.055556 | GO:0010951 | GO:BP | negative regulation of endopeptidase activity |
| P1 | 0.000456 | 727 | 24 | 0.134078 | 0.033012 | GO:0002263 | GO:BP | cell activation involved in immune response |
| P1 | 0.000832 | 265 | 14 | 0.078212 | 0.05283 | GO:0010466 | GO:BP | negative regulation of peptidase activity |
| P1 | 0.001974 | 3 | 3 | 0.01676 | 1 | GO:0033693 | GO:BP | neurofilament bundle assembly |
| P1 | 0.001974 | 3 | 3 | 0.01676 | 1 | GO:0099185 | GO:BP | postsynaptic intermediate filament cytoskeleton |
| | | | | | | | | organization |
| P1 | 0.002272 | 1222 | 31 | 0.173184 | 0.025368 | GO:0098542 | GO:BP | defense response to other organism |
| P1 | 0.002783 | 109 | 9 | 0.050279 | 0.082569 | GO:0030199 | GO:BP | collagen fibril organization |
| P1 | 0.002807 | 3457 | 61 | 0.340782 | 0.017645 | GO:0010033 | GO:BP | response to organic substance |
| P1 | 0.00328 | 1509 | 35 | 0.195531 | 0.023194 | GO:0022610 | GO:BP | biological adhesion |
| P1 | 0.003399 | 756 | 23 | 0.128492 | 0.030423 | GO:0030162 | GO:BP | regulation of proteolysis |
| P1 | 0.004229 | 2544 | 49 | 0.273743 | 0.019261 | GO:0006955 | GO:BP | immune response |
| P1 | 0.005474 | 356 | 15 | 0.083799 | 0.042135 | GO:0045861 | GO:BP | negative regulation of proteolysis |
| P1 | 0.005485 | 3443 | 60 | 0.335196 | 0.017427 | GO:0070887 | GO:BP | cellular response to chemical stimulus |
| P1 | 0.00596 | 1413 | 33 | 0.184358 | 0.023355 | GO:0140352 | GO:BP | export from cell |
| P1 | 0.006089 | 456 | 17 | 0.094972 | 0.037281 | GO:0051346 | GO:BP | negative regulation of hydrolase activity |
| P1 | 0.006446 | 1351 | 32 | 0.178771 | 0.023686 | GO:0032940 | GO:BP | secretion by cell |
| | 0.006501 | 4829 | 76 | 0.424581 | 0.015738 | GO:0042221 | GO:BP | response to chemical |
| P1 | 0.006945 | 42 | 6 | 0.03352 | 0.142857 | GO:0050832 | GO:BP | defense response to fungus |
| P1 | 0.007946 | 1639 | 36 | 0.201117 | 0.021965 | GO:0009607 | GO:BP | response to biotic stimulus |
| P1 | 0.008143 | 1502 | 34 | 0.189944 | 0.022636 | GO:0007155 | GO:BP | cell adhesion |
| P1 | 0.011799 | 3114 | 55 | 0.307263 | 0.017662 | GO:0009605 | GO:BP | response to external stimulus |
| P1 | 0.012206 | 1600 | 35 | 0.195531 | 0.021875 | GO:0051707 | GO:BP | response to other organism |
| P1 | 0.012376 | 1601 | 35 | 0.195531 | 0.021861 | GO:0043207 | GO:BP | response to external biotic stimulus |
| P1 | 0.016974 | 956 | 25 | 0.139665 | 0.026151 | GO:0033993 | GO:BP | response to lipid |
| P1 | 0.018893 | 1492 | 33 | 0.184358 | 0.022118 | GO:0046903 | GO:BP | secretion |
| P1 | 0.019691 | 1851 | 38 | 0.212291 | 0.020529 | GO:0006508 | GO:BP | proteolysis |
| P1 | 0.022819 | 77 | 7 | 0.039106 | 0.090909 | GO:0031640 | GO:BP | killing of cells of other organism |
| P1 | 0.024643 | 3438 | 58 | 0.324022 | 0.01687 | GO:0002376 | GO:BP | immune system process |
| P1 | 0.027231 | 511 | 17 | 0.094972 | 0.033268 | GO:0050900 | GO:BP | leukocyte migration |
| P1 | 0.028869 | 1956 | 39 | 0.217877 | 0.019939 | GO:0006952 | GO:BP | defense response |
| P1 | 0.030662 | 1252 | 29 | 0.162011 | 0.023163 | GO:0051336 | GO:BP | regulation of hydrolase activity |
| P1 | 0.031443 | 362 | 14 | 0.078212 | 0.038674 | GO:0002237 | GO:BP | response to molecule of bacterial origin |
| P1 | 0.032703 | 2816 | 50 | 0.27933 | 0.017756 | GO:0009653 | GO:BP | anatomical structure morphogenesis |
| P1 | 0.035936 | 8000 | 107 | 0.597765 | 0.013375 | GO:0032501 | GO:BP | multicellular organismal process |
| P1 | 0.038339 | 56 | 6 | 0.03352 | 0.107143 | GO:0009620 | GO:BP | response to fungus |
| P1 | 0.042636 | 1768 | 36 | 0.201117 | 0.020362 | GO:0044419 | GO:BP | biological process involved in interspecies interaction |
| | | | | | | | | between organisms |

TABLE 3
AUC of individual biomarkers

| Protein | AUC | AUC (lower)* | AUC (upper)* |
|---|---|---|---|
| CAPNS1 | 0.82 | 0.68 | 0.96 |
| GNA11 | 0.82 | 0.66 | 0.97 |
| SRD5A2 | 0.81 | 0.67 | 0.95 |
| LDHB | 0.79 | 0.65 | 0.94 |
| WDR5 | 0.79 | 0.63 | 0.96 |
| NCDN | 0.81 | 0.67 | 0.96 |

*AUC (lower) and AUC (upper) refer to the lower and upper range of the 95% confidence interval of the area under the receiver operator curve (AUC).

TABLE 4
AUC of biomarker pairs

| Protein | Protein | | AUC | AUC |
|---|---|---|---|---|
| 1 | 2 | AUC | (lower)* | (upper)* |
| CAPNS1 | GNA11 | 0.87 | 0.73 | 1 |
| CAPNS1 | SRD5A2 | 0.81 | 0.66 | 0.95 |
| CAPNS1 | LDHB | 0.90 | 0.79 | 1 |
| CAPNS1 | WDR5 | 0.83 | 0.65 | 1 |
| CAPNS1 | NCDN | 0.80 | 0.65 | 0.95 |
| GNA11 | SRD5A2 | 0.85 | 0.72 | 0.98 |
| GNA11 | LDHB | 0.85 | 0.74 | 0.97 |
| GNA11 | WDR5 | 0.79 | 0.61 | 0.97 |
| GNA11 | NCDN | 0.85 | 0.71 | 0.98 |
| SRD5A2 | LDHB | 0.88 | 0.77 | 0.99 |
| SRD5A2 | WDR5 | 0.83 | 0.69 | 0.97 |
| SRD5A2 | NCDN | 0.84 | 0.71 | 0.97 |
| LDHB | WDR5 | 0.87 | 0.75 | 1 |
| LDHB | NCDN | 0.86 | 0.74 | 0.98 |
| WDR5 | NCDN | 0.88 | 0.75 | 1 |

*AUC (lower) and AUC (upper) refer to the lower and upper range of the 95% confidence interval of the area under the receiver operator curve (AUC).

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### CITATIONS FOR REFERENCES REFERRED TO IN THE SPECIFICATION

1. Ahmed HU, EI-Shater Bosaily A, Brown L C, et al. Diagnostic accuracy of multi-parametric MRI and TRUS biopsy in prostate cancer (PROMIS): a paired validating confirmatory study. Lancet. 2017; 389(10071):815-822. doi:10.1016/S0140-6736(16)32401-1
2. Eklund M, Jaderling F, Discacciati A, et al. MRI-Targeted or Standard Biopsy in Prostate Cancer Screening. N Engl J Med. July 2021. doi:10.1056/NEJMoa2100852
3. Klotz L, Chin J, Black P C, et al. Comparison of Multiparametric Magnetic Resonance Imaging-Targeted Biopsy With Systematic Transrectal Ultrasonography Biopsy for Biopsy-Naïve Men at Risk for Prostate Cancer: A Phase 3 Randomized Clinical Trial. JAMA Oncol. 2021; 7(4):534-542. doi:10.1001/jamaonco1.2020.7589
4. Panebianco V, Barchetti G, Simone G, et al. Negative Multi-parametric Magnetic Resonance Imaging for Prostate Cancer: What's Next? Eur Urol. 2018; 74(1):48-54. doi:10.1016/j.eururo.2018.03.007
5. Houlahan K E, Salmasi A, Sadun T Y, et al. Molecular Hallmarks of Multiparametric Magnetic Resonance Imaging Visibility in Prostate Cancer. Eur Urol. 2019; 76(1):18-23. doi:10.1016/j.eururo.2018.12.036
6. Chua M L K, Lo W, Pintilie M, et al. A Prostate Cancer “Nimbosus”: Genomic Instability and SChLAP1 Dysregulation Underpin Aggression of Intraductal and Cribriform Subpathologies. Eur Urol. 2017; 72(5):665-674. doi:10.1016/j.eururo.2017.04.034
7. Bhandari V, Hoey C, Liu L Y, et al. Molecular landmarks of tumour hypoxia across cancer types. Nat Genet. 2019; 51(2):308-318. doi:10.1038/s41588-018-0318-2
8. Pachynski R K, Kim E H, Miheecheva N, et al. Single-cell spatial proteomic revelations on the multiparametric MRI heterogeneity of clinically significant prostate cancer. Clin Cancer Res. 2021; 27(12):3478-3490. doi:10.1158/1078-0432.CCR-20-4217
9. Sinha A, Huang V, Livingstone J, et al. The Proteogenomic Landscape of Curable Prostate Cancer. Cancer Cell. 2019; 35(3):414-427.e6. doi:10.1016/j.cce11.2019.02.005
10. Rodriguez-Blanco G, Zeneyedpour L, Duijvesz D, et al. Tissue proteomics outlines AGR2 AND LOX5 as markers for biochemical recurrence of prostate cancer. Oncotarget. 2018;9(92):36444-36456. doi:10.18632/oncotarget.26342
11. Pachynski R K, Kim E H, Miheecheva N, et al. Single-cell Spatial Proteomic Revelations on the Multiparametric MRI Heterogeneity of Clinically Significant Prostate Cancer. Clin cancer Res an Off J Am Assoc Cancer Res. 2021; 27(12):3478-3490. doi:10.1158/1078-0432.CCR-20-4217
12. Salami S S, Kaplan J B, Nallandhighal S, et al. Biologic Significance of Magnetic Resonance Imaging Invisibility in Localized Prostate Cancer. JCO Precis Oncol. 2019;(3):1-12. doi:10.1200/po.19.00054
13. Warner E W, Yip S M, Chi K N, Wyatt A W. DNA repair defects in prostate cancer: impact for screening, prognostication and treatment. BJU Int. 2019; 123(5):769-776. doi:10.1111/bju.14576
14. Zafarana G, Ishkanian A S, Malloff C A, et al. Copy number alterations of c-MYC and PTEN are prognostic factors for relapse after prostate cancer radiotherapy. Cancer. 2012; 118(16):4053-4062. doi:10.1002/cncr.26729
15. Salmasi A, Said J, Shindel A W, et al. A 17-Gene Genomic Prostate Score Assay Provides Independent Information on Adverse Pathology in the Setting of Combined Multiparametric Magnetic Resonance Imaging Fusion Targeted and Systematic Prostate Biopsy. J Urol. 2018; 200(3):564-572. doi:10.1016/j.juro.2018.03.004
16. Purysko A S, Magi-Galluzzi C, Mian O Y, et al. Correlation between MRI phenotypes and a genomic classifier of prostate cancer: preliminary findings. Eur Radiol. 2019;29(9):48614870. doi:10.1007/s00330-019-06114-x
17. Chen S, Huang V, Xu X, Bristow R G, Boutros PC. Widespread and Functional RNA Circularization in Localized Prostate Cancer. Cell. 2019; 176(4):831-843. doi:10.1016/j.ce11.2019.01.025
18. Wojtowicz E E, Lechman E R, Hermans K G, et al. Ectopic miR-125a Expression Induces Long-Term Repopulating Stem Cell Capacity in Mouse and Human Hematopoietic Progenitors. Cell Stem Cell. 2016; 19(3):383-396. doi:10.1016/j.stem.2016.06.008
19. Tyanova S, Temu T, Sinitcyn P, et al. The Perseus computational platform for comprehensive analysis of (prote)omics data. Nat Methods. 2016; 13(9):731-740. doi:10.1038/nmeth.3901
20. Ragnum H B, Vlatkovic L, Lie A K , et al. The tumour hypoxia marker pimonidazole reflects a transcriptional programme associated with aggressive prostate cancer. Br J Cancer. 2015; 112(2):382-390. doi:10.1038/bjc.2014.604
21. Subramanian A, Tamayo P, Mootha V K, et al. Gene set enrichment analysis: a knowledge-based approach for interpreting genome-wide expression profiles. Proc Natl Acad Sci USA. 2005; 102(43):15545-15550. doi:10.1073/pnas.0506580102
22. Mootha V K, Lindgren C M, Eriksson K-F, et al. PGC-1alpha-responsive genes involved in oxidative phosphorylation are coordinately downregulated in human diabetes. Nat Genet. 2003; 34(3):267-273. doi:10.1038/ng1180
23. Liberzon A, Birger C, Thorvaldsdottir H, Ghandi M, Mesirov J P, Tamayo P. The Molecular Signatures Database (MSigDB) hallmark gene set collection. Cell Syst. 2015; 1(6):417-425. doi:10.1016/j.cels.2015.12.004.
24. Lehallier et al. Undulating changes in human plasma proteome profiles across the lifespan. Nature Medicine 35, 1843-1850 (2019).

1. A method comprising:
(A) obtaining a sample collected from a subject; and measuring a polypeptide level of one or more mpMRI visibility biomarkers selected from SRD5A2, GNA11, CAPNS1, NCDN, WDR5 and/or LDHB, in the sample collected from the subject; or
(B) obtaining expression data and determining a polypeptide level of one or more mpMRI visibility biomarkers selected from SRD5A2, GNA11, CAPNS1, NCDN, WDR5 and/or LDHB, in a sample collected from a subject, and
selecting the subject for mpMRI if the level of the one or more mpMRI visibility biomarkers is indicative that the tumor is visible,
wherein the subject has or is suspected of having prostate cancer.

2. The method of claim 1, further comprising comparing the level of the one of more mpMRI visibility biomarkers to a predetermined threshold level or statistical model.

3. The method of claim 2, wherein the predetermined threshold level is determined from a plurality of normal tissue adjacent to tumour (NAT) samples.

4. The method of claim 1, wherein the one or more mpMRI visibility biomarkers are at least 2, 3, 4, or 5 of the mpMRI visibility biomarkers or wherein the one or more mpMRI visibility biomarkers are all of the mpMRI visibility biomarkers.

5-8. (canceled)

9. The method of claim 1, wherein the one or more mpMRI visibility biomarkers is or comprises SRD5A2, GNA11, CAPNS1, NCND, WDR5, or LDHB.

10-14. (canceled)

15. The method of claim 1, wherein the one or more mpMRI visibility biomarkers is or comprises SRD5A2, GNA11, and LDHB.

16. The method of claim 1, wherein the one or more mpMRI visibility biomarkers is or comprises SRD5A2 and GNA11; SRD5A2 and LDHB, GNA11 and LDHB, CAPNS1 and GNA11, CAPNS1 and SRD5A2, CAPNS1 and LDHB, CAPNS1 and WDR5, CAPNS1 and NCDN, GNA11 and WDR5, GNA11 and NCDN, SRD5A2 and WDR5, SRDSA and NCDN, LDHB and WDR5, LDHB and NCDN, or WDR5 and NCDN.

17-30. (canceled)

31. The method of claim 1, wherein the level is the log 2 transformed abundance of the biomarker.

32. The method of claim 2, wherein the statistical model comprises one or more of logistic regression, linear discriminant analysis, multivariate adaptive regression splines, naïve Bayes, neural network, support vector machine, functional tree, LAD tree, Bayesian network, elastic net regression, and random forest.

33. The method of claim 32, wherein the cutoff value is 0.5.

34. The method of claim 1, wherein the subject is a subject with grade 1 or grade 2 prostate cancer as defined by ISUP.

35. The method of claim 1, wherein the sample is a prostate cancer biopsy.

36. The method of claim 35, wherein the sample is a treatment-naïve sample.

37. The method of claim 35, wherein the sample is taken from a treatment-naïve subject.

38. The method of claim 1, wherein the level of the one or more biomarkers is measured by IHC or ELISA.

39. (canceled)

40. The method of claim 1, wherein the method further comprises performing mpMRI.

41. The method of claim 1, wherein the method further comprises repeating the method after an interval.

42-44.

45. The method of claim 1, further comprising providing a prognosis or an assessment of aggressiveness of the tumour to the subject.

46. The method of claim 45, further comprising selecting a suitable treatment plan and/or risk stratification.

47. A kit comprising at least two detection agents, each specific for a polypeptide selected from SRD5A2, GNA11, CAPNS1, NCDN, WDR5 and/or LDHB.
